(19) United States
(12) Patent Application Publication
(10) Pub. No.: US 2004/0186113 A1
(43) Pub. Date: Sep. 23, 2004

(54) Heterocyclic amines for the treatment of conditions associated with gsk-3

(76) Inventors: Stefan Berg, Sodertalje (SE); Sven Hellberg, Sodertalje (SE)

Correspondence Address: WHITE & CASE LLP, PATENT DEPARTMENT, 1155 AVENUE OF THE AMERICAS, NEW YORK, NY, 10036, US

(21) Appl. No.: 10/481,699

(22) PCT Filed: Jul. 3, 2002
(86) PCT No.: PCT/SE02/01340

(30) Foreign Application Priority Data
Jul. 5, 2001 (SE) 0102438-9

(51) Int. Cl. A61K 31/4965; A61K 31/4439; A61K 31/44; C 07D 4 3/02

(52) U.S. Cl. 514/255.05; 514/255.06; 514/340; 514/352; 544/405; 544/406; 546/268.1

(57) ABSTRACT

The present invention relates to new compounds of the formula (I) wherein Y, X, P, Q, R₁, R₂, R₃, R₄, R_{5A}, R₆, R₇, R₈, R₉, A, B, n, m are defined as in claim 1, a process for their preparation, pharmaceutical formulations containing said therapeutically active compounds and to the use of said active compounds for the treatment of conditions associated with glycogen synthase kinase-3 (GSK3) as well as an intermediate used in the preparation of said compounds.

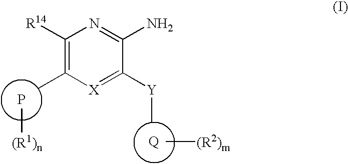

### FIELD OF THE INVENTION

The present invention relates to new compounds of the formula I, as a free base or a pharmaceutically acceptable salt thereof, to pharmaceutical formulations containing said compounds and to the use of said compounds in therapy. The present invention further relates the process for the preparation of compounds of the formula I and to a new intermediate prepared therein.

An object of the invention is to provide compounds of formula I for therapeutic use, especially compounds that are useful for the prevention and/or treatment of conditions associated with glycogen synthase kinase-3 (GSK3) in mammals including man. Particularly compounds of formula I exhibiting inhibition of GSK-3.

It is also an object of the invention to provide compounds with a therapeutic effect after oral administration.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed-in the central and peripheral nervous system. GSK3 phosphorylates several substrates including tau, β-catenin, glycogen ynthase, pyruvate dehydrogenase and elongation initiation factor 2b (eIF2b). Insulin and growth factors activate protein kinase B, which phosphorylates GSK3 on serine 9 residue and inactivates it.

Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but believed to be related. Glycogen synthase kinase 3β (GSK3β) or Tau (τ) phosphorylating kinase selectively phosphorylates the microtubule associated protein τ in neurons at sites that are hyperphosphorylated in AD brains. Hyperphosphorylated protein τ has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of τ and a paired helical filaments-like state via induction of GSK3β activity, followed by disruption of axonal transport and neuronal death (Inahori and Uchida, J. Biochem 121:179-188, 1997). GSK3β preferentially labels neurofibrillary tangles and has been shown to be active in pre-tangle neurons in AD brains. GSK3 protein levels are also increased by 50% in brain tissue from AD patients. Furthermore, GSK3βphosphorylates pyruvate dehydrogenase, a key enzyme in the glycolytic pathway and prevents the conversion of pyruvate to acetyl-Co-A (Hoshi et al., PNAS 93:2719-2723, 1996). Acetyl-Co-A is critical for the synthesis of acetylcholine, a neurotransmitter with cognitive functions. Thus, GSK3β inhibition may have beneficial effects in progression as well as the cognitive deficits associated with Alzheimer's disease and other above-referred to diseases.

Chronic and Acute Neurodegenerative Diseases.

Growth factor mediated activation of the PI3K /Akt pathway has been shown to play a key role in neuronal survival. The activation of this pathway results in GSK3β inhibition. Recent studies (Bhat et. al., PNAS 97:11074-11079 (2000)) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as Alzheimer's Disease, Parkinson's Disease, amyotrophic lateral sclerosis, Huntington's Disease and HIV dementia, ischemic stroke and head trauma. Lithium was neuroprotective in inhibiting apoptosis in cells and in the brain at doses that resulted in the inhibition of GSK3β. Thus GSK3β inhibitors could be useful in attenuating the course of neurodegenerative diseases.

Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The recent discovery that lithium inhibits GSK3 at therapeutic concentrations has raised the possibility that this enzyme represents a key target of lithium's action in the brain (Stambolic et al., Curr. Biol. 6:1664-1668, 1996; Klein and Melton; PNAS 93:8455-8459, 1996). Inhibition of GSK3β may therefore be of therapeutic relevance in the treatment of BD as well as in AD patients that have affective disorders.

Schizophrenia

GSK3 is involved in signal transduction cascades of multiple cellular processes, particularly during neural development. Kozlovsky et al (Am J Psychiatry May 2000; 157(5):831-3) found that GSK3β levels were 41% lowerin the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 9:1379-1383 (1998)).

Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also. over-expressed in muscles from Type II diabetic patients (Nikoulina et al., Diabetes. February 2000; 49(2):263-71). Inhibition of GSK3 increases the activity of glycogen synthase thereby decreasing glucose levels by its conversion to glycogen. GSK3 inhibition may therefore be of therapeutic relevance in the treatment of Type I and Type II diabetes and diabetic neuropathy.

Hair Loss

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell Nov. 25, 1998; 95 (5):605-14)). The new follicles formed sebaceous glands and dermal papilla, normally established only in embryogenesis. Thus GSK3 inhibition may offer treatment for baldness.

Oral contraceptives

Vijajaraghavan et al. (Biol Reprod June 2000; 62 (6):1647-54) reported that GSK3 is high in motile versus immotile sperm. Immunocytochemistry revealed that GSK3 is present in the flagellum and the anterior portion of the sperm head. These data suggest that GSK3 could be a key element underlying motility initiation in the epididymis and regulation of mature sperm function. Inhibitors of GSK3 could be useful as contraceptives for males.

### DISCLOSURE OF THE INVENTION

The object of the present invention is to provide compounds having a selective inhibiting effect at GSK3 as well as having a good bioavailability.

Accordingly, the present invention provides a compound of the formula I

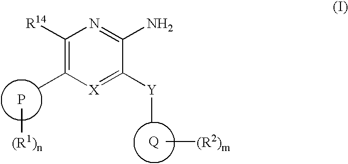

wherein:

Y is CONR³, NR³CO, SO₂NR³, NR³SO₂, CH₂NR³, NR³CH₂, NR³CONR³, C₁₋₆alkylene,

CH₂CO, COCH₂, CH═CH, OCH₂ or CH₂O;

X is CH or N;

P is phenyl or a 5 or 6 membered heteroaromatic ring containing one or more heteroatoms selected from N, O or S and said phenyl ring or 5 or 6 membered heteroaromatic ring may optionally be fused with a 5 or 6 membered saturated, partially saturated or unsaturated ring containing atoms selected from C, N, O or S;

Q is phenyl or a 5 or 6 membered heteroaromatic ring containing one or more heteroatoms selected from N, O or S wherein at least one atom is nitrogen;

R¹ is halo, nitro, C₀₋₆alkylCN, C₀₋₆alkylOR⁸, fluoromethyl, difluoromethyl, trifluoromethyl, C₀₋₆alkylNR⁸R⁹, C₀₋₆alkylCONR⁸R⁹, C₀₋₆alkylNR⁸(CO)R⁹, NR⁸(CO)OR⁹, C₀₋₆alkylO(CO)R⁸, C₀₋₆alkylSO₂R⁸, C₀₋₆alkylSOR⁸, C₀₋₆alkylCOR⁸, CO₀₋₆alkylO(CO)OR⁸, C₁₋₆alkylCO₂R⁸, OC₀₋₆alkylSO₂R⁸, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, CO alkylaryl or C₀₋₆alkylheteroaryl may be optionally substituted by one or more A;

R² is halo, nitro, CHO, C₀₋₆alkylCN, OC₁₋₆alkylCN, CO-₆alkylOR⁴, OC₁₋₆alkylOR⁴, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R⁵, OC₁₋₆alkylOC₁₋₆alkylNR⁴R⁵, NR⁴OR⁵ C₀₋₆alkylCO₂R⁴, OC₁₋₆alkylCO₂R⁴, C₀₋₆alkylCONR⁴R⁵, OC₁₋₆alkylCONR⁴R⁵, OC₁₋₆alkyNR⁴(CO)R⁵, C₀₋₆alkylNR⁴(CO)R⁵, O(CO)NR⁴R⁵, NR⁴(C)OR⁵, NR⁴(CO)NR⁴R⁵, O(CO)OR⁴, O(CO)R⁴, OC₁₋₆alkylCOR⁴, NR⁴(CO)(CO)R⁴, NR⁴(CO)(CO)NR⁴R⁵, SR⁴, C₀₋₆alkyl(SO₂)NR⁴R⁵, OC₁₋₆alkylNR⁴(SO₂)R⁵, OC₀₋₆alkyl(SO₂)NR⁴R⁵, C₀₋₆alkyl(SO)NR⁴R⁵, OC₁₋₆alkyl(SO)NR⁴R⁵, SO₃R⁴, C₁₋₆alkylNR⁴(SO₂)NR⁴R⁵, C₀₋₆alkylNR⁴(SO)R⁵, OC₀₋₆alkylNR⁴(SO)R⁵, OC₀₋₆alkylSO₂R⁴, C₀₋₆alkylSO₂R⁴, C₀₋₆alkylSOR⁴, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted by one or more A;

m is 0, 1, 2, 3 or 4;

n is 0, 1, 2, 3, 4 or 5;

R³ is hydrogen, C₁₋₆alkyl, C₂₋₆alkenyl, C₂₋₆alkynyl, C₀₋₆alkylC₃₋₆cycloalkyl, C₁₋₆alkylNR⁶R⁷ or C₁₋₆alkylCONR⁶R⁷;

R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁶R⁷;

R⁴ and R⁵ may together form a 5 or 6 membered heterocyclic ring containing one or more heteroatoms selected from N, O or S, wherein said heterocyclic ring may be optionally substituted by A;

R⁶ and R⁷ are independently selected from hydrogen, C₁₋₆alkyl, C₂₋₆alkenyl, C₂₋₆alkynyl and C₀₋₆alkylC₃₋₆cycloalkyl,

R⁶ and R⁷ may together form a 5 or 6 membered heterocyclic ring containing one or more heteroatoms selected from N, O or S, wherein said heterocyclic ring may be optionally substituted by A;

R⁸ and R⁹ are independently selected from hydrogen, C₁₋₆alkyl, C₂₋₆alkenyl, C₂₋₆alkynyl, C₀₋₆alkylaryl, C₀₋₆alkylheteroaryl and C₀₋₆alkylC₃₋₆cycloalkyl;

R⁸ and R⁹ may together form a 5 or 6 membered heterocyclic ring containing one or more heteroatoms selected from N, O or S, wherein said heterocyclic ring may be optionally substituted by A;

R¹⁴ is hydrogen, methyl, fluoro, chloro or bromo;

wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl defined under R³ to R⁹ may be substituted by one-or more A;

A is halo, nitro, CHO, CN, OR⁴, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R⁵, NR⁴R⁵, CO₂R⁸, CONR⁴R⁵, NR⁴(CO)R⁴, O(CO)R⁴, COR⁴, SR⁴, (SO₂)NR⁴R⁵, (SO)NR⁴R⁵, SO₃R⁴, SO₂R⁴ or SOR⁴, as a free base or a pharmaceutically acceptable salt thereof, with the proviso that

Y is not methylene or ethylene when both P and Q are phenyl and

Y is not methylene when P is methoxypyrazine and Q is phenyl.

One aspect of the invention relates to a compound of formula I

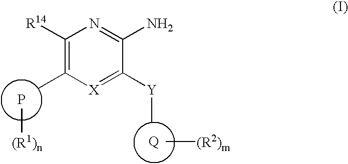

wherein:

Y is CONR³; NR³CO, SO₂NR³, NR³SO₂, CH₂NR³, NR³CH₂, NR³CONR³, CH₂CO, COCH₂, CH═CH, OCH₂ or CH₂O;

X is CH or N;

P is phenyl or a 5 or 6 membered heteroaromatic ring containing one or more heteroatoms selected from N, O or S and said phenyl ring or 5 or 6 membered heteroaromatic ring may optionally be fused with a 5 or 6 membered saturated, partially saturated or unsaturated ring containing atoms selected from C, N, O or S;

Q is phenyl or a 5 or 6 membered heteroaromatic ring containing one or more heteroatoms selected from N, O or S wherein at least one atom is nitrogen;

R¹ is halo, nitro, C₀₋₆alkylCN, C₀₋₆alkylOR⁸, fluoromethyl, difluoromethyl, trifluoromethyl, C₀₋₆alkylNR⁸R⁹, C₀₋₆alkylCONR⁸R⁹, C₀₋₆alkylNR⁸(CO)R^{9,} NR⁸(CO)OR⁹, C₀₋₆alkylO(CO)R⁸, C₀₋₆alkylSO₂R⁸, C₀₋₆alkylSOR⁸, C₀₋₆alkylCOR⁸, C₀₋₆alkylO(CO)OR⁸, OC₀₋₆alkylSO₂R⁸, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted on any carbon atom by one or more A; and if said heteroaryl contains a —NH— moiety that nitrogen may be optionally substituted by A;

R² is halo, nitro, CHO, C₀₋₆alkylCN, OC₀₋₆alkylCN, C₀₋₆alkylOR⁴, OC₁₋₆alkylOR⁴, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R⁵, OC₁₋₆alkylOC₁₋₆alkylNR⁴R⁵, NR^{4 OR}⁵ C₀₋₆alkylCO₂R⁴, OC₁₋₆alkylCO₂R⁴, C₁₋₆alkylCONR⁵, OC₁₋₆alkylCONR⁴R⁵, OC₁₋₆alkylNR⁴(CO)R⁵, C₀₋₆alkylNR⁴(CO)R⁵, O(CO)NR⁴R⁵, NR⁴(CO)OR⁵, NR⁴(CO)NR⁴R⁵, O(CO)OR⁴, O(CO)R⁴, OC₁₋₆alkylCOR⁴, NR⁴(CO)(CO)R⁴, NR⁴(CO)(CO)NR⁴R⁵, SR⁴, C₀₋₆alkyl(SO₂)NR⁴R⁵, OC₁₋₆alkylNR⁴(SO₂)R⁵, OC₀₋₆alkyl(SO₂)NR⁴R⁵, C₀₋₆alkyl(SO)NR⁴R⁵, OC₁₋₆alkyl(SO)NR⁴R⁵, SO₃R⁴, C₁₋₆alkylNR⁴(SO₂)NR⁴R⁵, C₀₋₆alkylNR⁴(SO)R⁵, OC₀₋₆alkylNR⁴(SO)R⁵, OC₀₋₆alkylSO₂R⁴, C₀₋₆alkylSO₂R⁴, C₀₋₆alkylSOR⁴, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted on any carbon atom by one or more A, and if said heteroaryl contains a —NH— moiety that nitrogen may be optionally substituted by A;

m is 0, 1, 2, 3 or 4;

n is 0, 1, 2, 3, 4 or 5;

R³ is hydrogen, C₁₋₆alkyl, C₂₋₆alkenyl, C₂₋₆alkynyl, C₀₋₆alkylC₃₋₆cycloalkyl, C₁₋₆alkylNR⁶R⁷ or C₁₋₆alkylCONR⁶R⁷;

R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁶R⁷;

R⁴ and R⁵ may together form a 5 or 6 membered heterocyclic ring containing one or more heteroatoms selected from N, O or S, wherein if said heterocyclic ring contains an —NH— moiety that ring nitrogen may be optionally substituted by A;

R⁶ and R⁷ are independently selected from hydrogen, C₁₋₆alkyl, C₂₋₆alkenyl, C₂₋₆alkynyl and C₀₋₆alkylC₃₋₆cycloalkyl;

R⁶ and R⁷ may together form a 5 or 6 membered heterocyclic ring containing one or more heteroatoms selected from N, O or S, and if said heterocyclic ring contains a —NH— moiety that ring nitrogen may be optionally substituted by A;

R⁸ and R⁹ are independently selected from hydrogen, C₁₋₆alkyl, C₂₋₆alkenyl, C₂₋₆alkynyl, C₀₋₆alkylaryl, C₀₋₆alkylheteroaryl and C₀₋₆alkylC₃₋₆cycloalkyl;

R⁸ and R⁹ may together form a 5 or 6 membered heterocyclic ring containing one or more heteroatoms selected from N, O or S, and if said heterocyclic ring contains a —NH— moiety that ring nitrogen may be optionally substituted by A;

R¹⁴ is hydrogen;

wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl defined under R³ to R⁹ may be substituted by one or more A;

A is halo, nitro, CHO, CN, OR⁴, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R⁵, NR⁴R⁵, CO₂R⁴, CONR⁴R⁵, NR⁴(CO)R⁴, O(CO)R⁴, COR⁴, SR⁴, (SO₂)NR⁴R⁵, (SO)NR⁴R⁵, SO₃R⁴, SO₂R⁴ or SOR⁴, as a free base or a pharmaceutically acceptable salt thereof.

Another aspect of the invention relates to compounds of formula I

wherein:

Y is CONR³;

X is N;

P is phenyl or a 5 membered heteroaromatic ring containing one heteroatom selected from

O or S and said phenyl ring or 5 or 6 membered heteroaromatic ring may optionally be fused with a 5 membered saturated ring containing atoms selected from C or O;

Q is pyridine;

R¹ is halo, nitro, C₀₋₆alkylCN, C₀₋₆alkylOR⁸, trifluoromethyl, C₀₋₆alkylCONR⁸R⁹, C₁₋₆alkyl, C₁₋₆alkylCO₂R⁸, C₀₋₆alkylOR⁴ or C₀₋₆alkylNR⁴R⁵;

m is 0 or 1;

n is 0, 1 or 2;

R³ is hydrogen;

R⁴ and R⁵ are hydrogen;

R⁴ and R⁵ may together form a 5 membered heterocyclic ring containing one heteroatom selected from N;

R⁸ and R⁹ are hydrogen;

R⁴ is hydrogen or methyl.

A preferred embodiment of the invention relates to compounds of formula I, wherein Y is CONR³.

In one aspect of the invention P is phenyl, furan, thiophene or another 5 or 6 membered heteroaromatic ring containing one or more heteroatoms selected from N, O or S.

In another aspect of the invention preferably Q is pyridine.

The invention further relates to compounds which are

3-Amino-6-phenyl-N-pyridin-3-ylpyrazine-2-carboxamide,

3-Amino-6-(2-methylphenyl)-N-pyridin-3-ylpyrazine-2-carboxamide,

3-Amino-6-(4-cyanophenyl)-N-pyridin-3-ylpyrazine-2-carboxamide,

3-Amino-6-(3,4-methylenedioxyphenyl)-N-pyridin-3-ylpytazine-2-carboxamide,

3-Amino-6-(2-thienyl)-N-pyridin-3-ylpyrazine-2-carboxamide,

3-Amino-6-(3-nitrophenyl)-N-pyridin-3-ylpyrazine-2-carboxamide,

3-Amino-6-(3,5-bistriflouromethylphenyl)-N-pyridin-3-ylpyrazine-2-carboxamide,

3-Amino-6-(3-thienyl)-N-pyridin-3-ylpyrazine-2-carboxamide,

3-Amino-6-(4-fluorophenyl)-N-pyridin-3-ylpyrazine-2-carboxamide,

3-Amino-6-(4-chlorophenyl)-N-pyridin-3-ylpyrazine-2-carboxamide,

3-Amino-6-(4-dichlorophenyl)-N-pyridin-3-ylpyrazine-2-carboxamide,

3-Amino-6-(2,3-dichlorophenyl)-N-pyridin-3-ylpyrazine-2-carboxamide,

3-Amino-6-(2,4-dichlorophenyl)-N-pyridin-3-ylpyrazine-2-carboxamide,

3-Amino-6-(2,4-difluorophenyl)-N-pyridin-3-ylpyrazine-2-carboxamide,

3-Amino-6-(3,4-difluorophenyl)-N-pyridin-3-ylpyrazine-2-carboxamide,

3-Amino-6-(3-chloro-4-fluorophenyl)-N-pyridin-3-ylpyrazine-2-carboxamide,

3-Amino-6-[4-fluoro-3-methylphenyl]-N-pyridin-3-ylpyrazine-2-carboxamide,

3-Amino-6-(3,4-dimethylphenyl)-N-pyridin-3-ylpyrazine-2-carboxamide,

3-Amino-6-(3-fluorophenyl)-N-pyridin-3-ylpyrazine-2-carboxamide and

3-Amino-6-(2-fluorophenyl)-N-pyridin-3-ylpyrazine-2-carboxamide as a free base or a pharmaceutically acceptable salt thereof.

The invention also relates to compounds,

3-Amino-6-(2,4-dichlorophenyl)-N-[4-(2-pyrrolidin-1-ylethyl)pyridin-3-yl]pyrazine-2-carboxamide,

3-Amino-6-(3-chloro-4-fluorophenyl)-N-[4-(2-pyrrolidin-1-ylethyl)pyridin-3-yl]pyrazine-2-carboxamide,

3-Amino-6-(2-furyl)-N-pyridin-3-ylpyrazine-2-carboxamide,

3-Amino-6-(4-hydroxyphenyl)-N-pyridin-3-ylpyrazine-2-carboxamide, and

3-Amino-6-[4-(aminocarbonyl)phenyl]-N-pyridin-3-ylpyrazine-2-carboxamide as a free base or a pharmaceutically acceptable salt thereof, and

3-Amino-6-(2,4-dichlorophenyl)-N-[4-(2-pyrrolidin-1-ylethyl)pyridin-3-yl]pyrazine-2-carboxamide hydrochloride.

3-Amino-6-(3-chloro-4-fluorophenyl)-N-[4-(2-pyrrolidin-1-ylethyl)pyridin-3-yl]pyrazine-2-carboxamide hydrochloride and

3-Amino-6-(4-hydroxyphenyl)-N-pyridin-3-ylpyrazine-2-carboxamide hydrochloride.

A further aspect of the invention relates to compounds,

3-Amino-6-(4-chlorophenyl)-5-methyl-N-pyridin-3-ylpyrazine-2-carboxamide and

4-{5-Amino-6-[(pyridin-3-ylamino)carbonyl]pyrazin-2-yl} benzoic acid as a free base or a pharmaceutically acceptable salt thereof.

Listed below are definitions of various terms used in the specification and claims to describe the present invention.

In this specification the term “alkyl” includes both straight and branched chain alkyl groups. The term C₁₋₆alkyl having 1 to 6 carbon atoms and may be methyl, ethyl, n-propyl, i-propyl, n-butyl, i-butyl, s-butyl, t-butyl, n-pentyl, i-pentyl, t-pentyl, neo-pentyl, n-hexyl or i-hexyl. The term C₁₋₃alkyl having 1 to 3 carbon atoms and may be methyl, ethyl, n-propyl or i-propyl. The term C₁₋₂alkyl having 1 to 2 carbon atoms and may be methyl or ethyl.

A similar convention applies to other radicals, for example “C₀₋₆alkylaryl” includes 1-phenylethyl and 2-phenylethyl.

In the case where a subscript is the integer 0 (zero) the group to which the subscript refers to indicates that the group is be absent, i.e. there is a direct bond between the groups.

The term “cycloalkyl” refers to an optionally substituted, saturated cyclic hydrocarbon ring system. The term “C₃₋₆cycloalkyl” may be cyclopropyl, cyclobutyl, cyclopentyl or cyclohexyl.

The term “alkenyl” refers to a straight or branched chain alkenyl group. The term C₂₋₆alkenyl having 2 to 6 carbon atoms and one double bond, and may be vinyl, allyl, propenyl, i-propenyl, butenyl, i-butenyl, crotyl, pentenyl, i-pentenyl or hexenyl. The term C₂₋₃alkenyl having 2 to 3 carbon atoms and one or two double bond, and may be vinyl, allyl, propenyl or i-propenyl.

The term “alkynyl” refers to a straight or branched chain alkynyl groups. The term C₂₋₆alkynyl having 2 to 6 carbon atoms and one trippel bond, and may be etynyl, propargyl, butynyl, i-butynyl, pentynyl, i-pentynyl or hexynyl. The term C₂₋₃alkynyl having 2 to 3 carbon atoms and one tripper bond, and may be etenyl or propargyl.

The term “halo” refers to fluoro, chloro, bromo and iodo.

The term “aryl” refers to an optionally substituted monocyclic or bicyclic hydrocarbon ring system containing at least one unsaturated aromatic ring. The “aryl” may be fused with a C₅₋₇ cycloalkyl ring to form a bicyclic hydrocarbon ring system. Examples and suitable values of the term “aryl” are phenyl, naphthyl, indanyl or tetralinyl.

The term “heteroaryl” and “5 or 6 membered heteroaromatic ring” containing one or more is heteroatoms selected from N, O and S may be furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl.

The term “heterocyclic ring” containing one or more heteroatoms selected from N, O or S may optionally contain a carbonyl function and is preferably a 5 or 6 membered heterocyclic ring and may be imidazolidinyl, imidazolinyl, morpholinyl, piperazinyl, piperidinyl, piperidonyl, pyrazolidinyl, pyrazolinyl, pyrrolidinyl, pyrrolinyl, tetrahydropyranyl, thiomorpholinyl. In the case where the heterocyclic ring contains a heteroatom selected from S this includes optionally SO and SO₂.

It is to be understood that when m is greater than one, R¹ groups may be the same or different. Similarly when m is greater than one the R² groups may be the same or different.

The term “hydrochloride” includes monohydrochloride, hydrochloride and hydrochloride salts.

A suitable pharmaceutically acceptable salt of the compounds of the invention is, for example, an acid-addition salt, which is sufficiently basic, for example an inorganic or organic acid. In addition, a suitable pharmaceutically acceptable salt of the compounds of the invention, which is sufficiently acidic is an alkali metal salt, an alkaline earth metal salt or a salt with an organic base, which affords a physiologically-acceptable cation.

Some compounds of the formula I may have chiral centres and/or geometric isomeric centres (E- and Z-isomers), and it is to be understood that the invention encompasses all such optical, diastereoisomers and geometric isomers.

The invention relates to any and all tautomeric forms of the compounds of the formula I.

An aspect of the present invention relates to a compound of formula VI

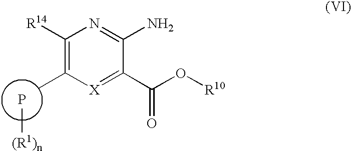

wherein X, P, R¹, R⁸, R⁹, R¹⁴, A and n are defined as in formula I according to any one of claims 1 to 3 and R¹⁰ is hydrogen or C₁₋₆alkyl, with the proviso that

i) when P is phenyl then R¹⁰ is C₃₋₆alkyl;

ii) when P is 4-chlorophenyl then R¹⁰ is C₂₋₆alkyl;

iii) when P is 4-methoxyphenyl then R¹⁰ is hydrogen or C₂₋₆alkyl;

iv) when P is pyridine then R¹⁰ cannot be methyl, ethyl or n-butyl;

v) when P is furan or benzothienyl then R¹⁰ cannot be methyl.

The invention further relates to compounds of formula VI, wherein P is phenyl and R¹⁰ is C₃₋₆alkyl.

The invention also relates to compounds of formula VI, wherein P is furan and R¹⁰ is C₂₋₆alkyl.

The invention even further relates to compounds of formula VI, wherein P is thiophene.

Another aspect of the present invention is a compound of formula IV

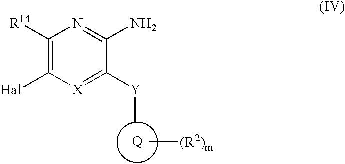

wherein X, R², R⁴, R⁵, R⁶, R⁷, A and m are defined as in formula I and R¹⁴ is hydrogen or methyl.

A further aspect of the present invention are compounds

3-Amino-6-bromo-N-pyridin-3-ylpyrazine-2-carboxamide,

3-Amino-6-bromo-5-methyl-N-pyridin-3-ylpyrazine-2-carboxamide,

tert-Butyl 4-(2-hydroxyethyl)pyridin-3-ylcarbamate,

tert-Butyl 4-(2-pyrrolidin-1-ylethyl)pyridin-3-ylcarbamate,

4-(2-Pyrrolidin-1-ylethyl)pyridin-3-amine and

3-Amino-6-bromo-N-[4-(2-pyrrolidin-1-ylethyl)pyridin-3-yl]pyrazine-2-carboxamide.

Methods of Preparation

Another aspect of the present invention provides a process for preparing a compound of formula I as a free base or a pharmaceutically acceptable salt thereof. Throughout the following description of such processes it is understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for examples in “Protective Groups in Organic Synthesis” T. W. Green, P. G. M. Wuts, Wiley-Interscience, New York, 1999.

Methods of Preparation of the Intermediates.

The processes for the preparation of the intermediates, wherein Y, X, P, Q, R¹, R², R³, R⁴, R⁵, R⁶, R⁷, R⁸, R⁹, R¹⁴, A, m and n are, unless specified otherwise, defined as in formula I, comprises of:

(i) reacting of a compound of formula II, wherein X is N or CH, R¹⁰ is hydrogen, C₁₋₆alkyl or when R¹⁰ is hydrogen in the form of a salt such as a sodium salt:

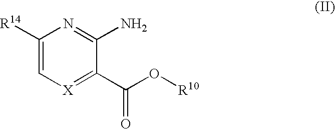

with a suitable halogenating reagent such as iodine, bromine or chlorine, halide salts such as ICl, BrCl or HOCl or other suitable halogenation reagents such as N-bromosuccinimide or phosphorous tribromide to obtain a compound of formula III. The reaction may be catalysed by metals or acids such as Fe, Cu-salts, acetic acid or sulfuric acid or aided by oxidising agents such as nitric acid, hydrogen peroxide or sulfur trioxide. The reaction may be carried out in a suitable solvent such as water, acetic acid or chloroform at a temperature in the range of −70° C. to +100° C.

(iia) amidation of a compound of formula II, wherein X is N or CH, R¹⁰ is C₁₋₆alkyl and R¹⁴ are as defined above:

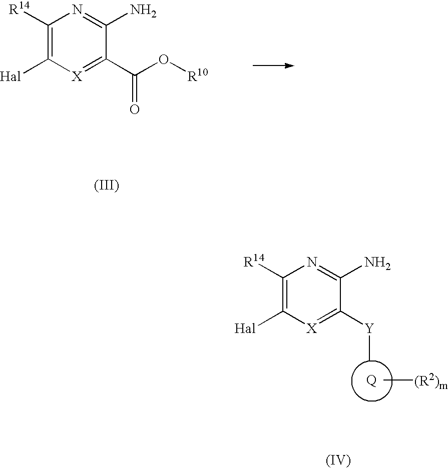

to obtain a compound of formula IV, wherein Q, R² and m are as defined above and Y is CONR³ may be carried out by treating a compound of formula III with the appropriate amine such as a compound of formula XI or 3-aminopyridine. The reaction can be performed neat or using a suitable solvent such as N,N-dimethylformamide, methylene chloride or ethyl acetate at a temperature ranging from −25° C. to +150° C. The reaction may be aided by using a base such as potassium carbonate, triethylamine or 1,8-diazabicyclo[5.4.0]undec-7-ene or an acid such as trimethylaluminum or p-toulenesulfonic acid.

(iib) amidation of a compound of formula III, wherein R¹⁰ is hydrogen, to obtain a compound of formula IV, may be performed by activation of the carboxylic acid function of a compound of formula III by treating the compound with coupling reagents such as 1-[3-(dimethylamino)propyl]-3-ethylcarbodiimide hydrochloride and 1-hydroxybenzotriazole hydrate, 1,3-dicyclohexylcarbodiimide and 1-hydroxybenzotriazole hydrate, 1,1′-carbonyldiimidazole or O-(7-azabenzotriazol-1-yl)-N,N,N′,N′-tetramethyluronium hexafluorophosphate or using an acyl halide reagent such as cyanuric chloride, oxalyl chloride, thionyl chloride or bromotrispyrrolidinophosphonium hexafluorophosphate followed by treatment with the appropriate amine such as a compound of formula XI or 3-aminopyridine.

(iii) amidation of a compound of formula II, wherein X and R¹⁴ are as defined above and R¹⁰ is hydrogen or C₁₋₆alkyl, to obtain a compound of formula V, may be carried out by amidation conditions described in (iia) and (iib) above to obtain a compound of formula V, wherein Y is CONR³ and R² and R¹⁴ are a substituent that is not susceptible to certain coupling agents;

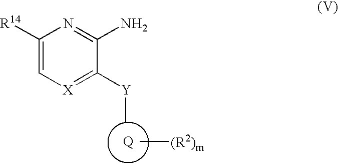

followed by,

halogenation of a compound of formula V with a halogenating reagent as described in (i) above to obtain a compound of formula IV.

(iv) conversion of a compound of formula III to a compound of formula VI, wherein X and R¹⁴ are as defined above and R¹⁰ is C₁₋₆alkyl, may be carried out by a de-halogen coupling with a suitable aryl:

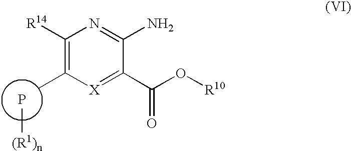

the reaction may be carried out by coupling of a compound of formula III with

a) an aryl halide such as aryl iodide, aryl bromide or aryl chloride in the presence of a metal such as copper, nickel, zinc and nickel complexes, copper oxide or palladium acetate and tetrabutylammonium bromide and a base such as potassium carbonate or an alkyl amine such as triethylamine. The reaction may occur between +20° C. and +180° C. in a suitable solvent such as N,N-dimethylformamide, toluene or 2-pentanol;

or,

b) an aryl boronic acid or a boronic ester. The reaction may be carried out using a'suitable palladium catalyst such as Pd(PPh₃)₄, Pd(dppf)Cl₂ or Pd(OAc)₂ together with a suitable ligand such as P(tert-butyl)₃ or 2-(dicyclohexylphosphino)biphenyl or a nickel catalyst such as nickel on charcoal of Ni(dppe)Cl₂ together with Zn and sodium triphenylphosphinetrimetasulfonate. A suitable base such as potassium carbonate, sodium hydroxide or cesium fluoride may be used in the reaction, which may be performed in a temperature range between +20° C. and +120° C. in a suitable solvent such as toluene, tetrahydrofuran or N,N-dimethylformamide;

or,

c) an aryl stannane in the presence of palladium catalyst such as Pd(PPh₃)₄, Pd(PPh₃)₂Cl₂ or Pd(dba)₃, with or without a reagent such as 4-tert-butylcatechole, lithium chloride or potassium carbonate. Suitable solvents maybe toluene, tetrahydrofuran or N,N-dimethylformamide. The reaction may occur in the temperature range of +20° C. and +120° C.

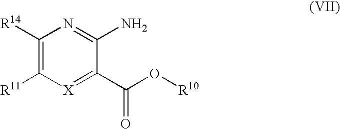

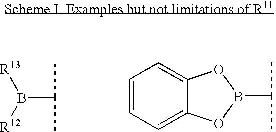

(v) conversion of a compound of formula VII, wherein X, R¹⁰ and R¹⁴ are as defined above and R¹¹ is a group outlined in Scheme I, wherein R¹² and R¹³ are C₁₋₆alkyl or C₁₋₃alkyl fused together to form a 5 or 6 membered boron-oxygen-C₂₋₃cycloalkyl and the alkyl, cycloalkyl and the aryl moieties may be optionally substituted, to obtain a compound of formula VI may be carried out by reacting a compound of formula VII with a suitable aryl halide. The reaction may be carried out using a suitable palladium catalyst such as Pd(PPh₃)₄, Pd(dppf)Cl₂ or Pd(OAc)₂ together with a suitable ligand, or a nickel catalyst such as nickel on charcoal or Ni(dppe)Cl₂ together with Zn and sodium triphenylphosphinetrimetasulfonate. A suitable base such as potassium carbonate, sodium hydroxide or cesium fluoride may be used in the reaction, which may be performed in a temperature range between +20° C. and +120° C. in a suitable solvent such as toluene, tetrahydrofuran or N,N-dimethylformamide.

(vi) borylation of a compound of formula III to a compound of formula VII, wherein X is N or CH, R¹⁰ and R¹⁴ are as defined above and R¹¹ may be a group outlined in Scheme I, wherein R¹² and R¹³ are C₁₋₆alkyl or C₁-alkyl fused together to form a 5 or 6 membered boron-oxygen-C₂₋₃cycloalkyl and the alkyl, cycloalkyl and the aryl moieties may be optionally substituted, may be carried out by a reaction with:

a) butylithium or magnesium and a suitable boron compound such as trimethyl borate or triisopropyl borate. The reaction may be performed in a suitable solvent such as tetrahydrofuran, hexane or methylene chloride in a temperature range between −100° C. and +20° C.;

or,

b) a palladium catalyst such as palladium tetrakistriphenylphosphine, palladium diphenylphosphineferrocene dichloride or palladium acetate together with a ligand such as 2-(dicyclohexylphosphino)biphenyl and a suitable boron species such as biscatecholatodiboron, bispinacolatodiboron or pinacolborane. A suitable base, which under the reaction conditions does not promote dimerisation of a compound of formula m, such as a tertiary amine such as trietylamine or diisopropylethylamine, or potassium is acetate may be used. The reaction may be performed in a solvent such as dioxane, toluene or acetonitrile at temperatures between +80° C. and +100° C.

(vii) borylation of a compound of formula IV to obtain a compound of formula VIII, wherein X, R², R¹¹, R¹⁴ and m are as defined above and Y is CONR³, may be carried out by the reaction conditions described in (vi):

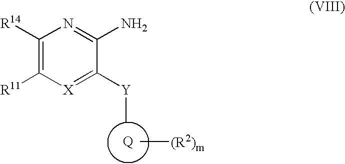

(viii) amidation of a compound of formula VII, wherein X is N or CH, R¹⁰ is C₁₋₆alkyl and R¹¹ is above, to obtain a compound of formula VIII, wherein X, R², R¹¹, R¹⁴ and m are as defined above and Y is CONR³ may be carried out by reacting a compound of formula VII with a suitable amine such as a compound of formula XI or 3-aminopyridine, under reaction conditions described in (iia) and (iib).

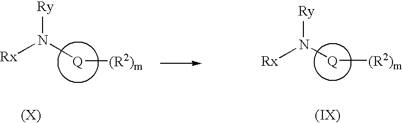

(ix) reacting a compound of formula X, wherein Q is a pyridine ring, R² is hydrogen (when m=0), bromine or iodide, m is 1 and wherein at least one of Rx or Ry is a suitable protecting group CO₂R⁸ to form a carbamate such as tert-butyl carbamate and the other of the Rx or Ry (in the case of one protecting group) is hydrogen, to obtain a compound of formula IX, wherein Q is a pyridine ring, R² is C₁₋₆alkylR⁴R⁵ and m is 1, may be carried out by reaction with butyllithium in a suitable solvent such as tetrahydrofuran or hexane followed by the addition of a suitable reagent such as ethylene oxide followed by the activation of the formed alcohol by the formation of the mesylate or the tosylate with a suitable reagent such as methansulfonyl chloride or para-toluensulfonyl chloride in a Is suitable solvent such as methylene chloride or tetrahydrofuran with or without a suitable base such as potassium carbonate or a trialkyl amine such as triethyl amine and at a suitable reaction temperature range between 0° C. and +100° C. followed by the addition of the appropriate amine HNR⁴R⁵ at a reaction temperature range between 0° C. and +100° C.

(x) hydrolysis of a compound of formula IX, to obtain a compound of formula XI,

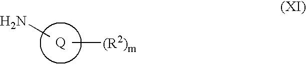

wherein Q is as defined above, R² is C₁₋₆alkylNR⁴R⁵ and m is 1, may be carried out by treating a compound of formula IX under acidic conditions using suitable acids such as hydrochloric acid or trafluoroacetic acid neat or in an appropriate solvent such as methanol, acetonitrile, methylene chloride or tetrahydrofuran and at a temperature interval between 0° C. and +80° C.

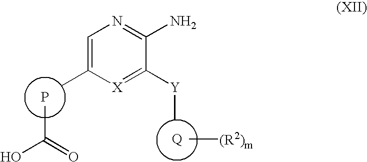

(xi) conversion of a compound of formula IV, to obtain a compound of formula XII, wherein X, P, Q, Y, R² and m are as defined above, may be carried out by a de-halogen coupling, wherein R² is a substituent that is not susceptible to certain agents in the reaction, of a compound of formula IV with an appropriate aryl boronic acid or a boronic ester. The reaction may be carried out using a suitable palladium catalyst such as Pd(PPh₃)₄, Pd(dppf)Cl₂ or Pd(OAc)₂ with or without a suitable ligand such as P(tert-butyl)₃ or 2-(dicyclohexylphosphino)biphenyl, or a nickel catalyst such as nickel on charcoal or Ni(dppe)Cl₂ together with Zn and sodium triphenylphosphinetrimetasulfonate. A suitable base such as potassium carbonate, sodium carbonate, sodium hydroxide or cesium fluoride may be used in the reaction, which is performed in the temperature range between +20° C. and +160° C. using an oil bath or in a microwave oven in a suitable solvent or solvent mixture such as toluene, tetrahydrofuran, dimethoxyethane/water or N,N-dimethylformamide.

Methods of Preparation of End Products

Another object of the invention are processes for the preparation of a compound of formula I, wherein Y, X, P, Q, R¹, R², R³, R⁴, R⁵, R⁶, R⁷, R⁸, R⁹, R¹⁴, A, n and m are, unless specified otherwise, defined as in formula I, comprising of:

A de-halogen coupling of a compound of formula IV with a suitable aryl species to give a compound of formula I:

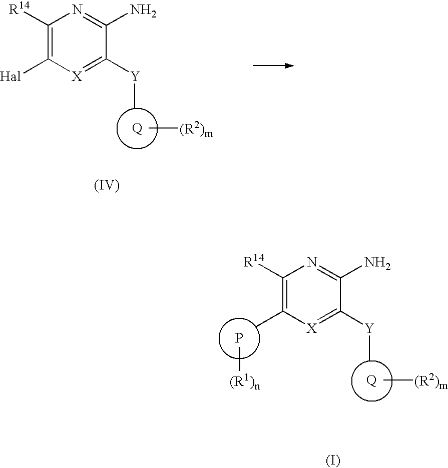

Thus, the de-halogen coupling according to process A may be carried out by coupling of a compound of formula IV with:

a) the appropriate aryl halogen such as aryl iodide, aryl bromide or aryl chloride in the presence of metals such as copper, nickel, zinc and nickel complexes, copper oxide or palladium acetate and tetrabutylammonium bromide and a base such as potassium carbonate or triethylamine. The reaction may occur between +20° C. and +180° C. in a suitable solvent such as N,N-dimethylformamide, toluene or 2-pentanol;

or,

b) an aryl boronic acid or a boronic ester. The reaction may be carried out using a suitable palladium catalyst such as Pd(PPh₃)₄, Pd(dppf)Cl₂ or Pd(OAc)₂ with or without a suitable ligand such as P(tert-butyl)₃, 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g triethyl amine, or potassium carbonate, sodium hydroxide or cesium fluoride may be used in the reaction, which is performed in the temperature range between +20° C. and +120° C. in a suitable solvent such as toluene, tetrahydrofuran or N,N-dimethylformamide;

or,

c) an aryl stannane in the presence of palladium catalyst such as. Pd(PPh₃)₄, Pd(PPh₃)₂Cl₂ or Pd(dba)₃, and if needed a helping reagent such as 4-tert-butylcatechole, lithium chloride or potassium carbonate. Suitable solvents may be toluene, tetrahydrofuran or N,N-dimethylformamide. The reaction may occur in a temperature range of +20° C. and +120° C.

B amidation of a compound of formula VI with the appropriate amine:

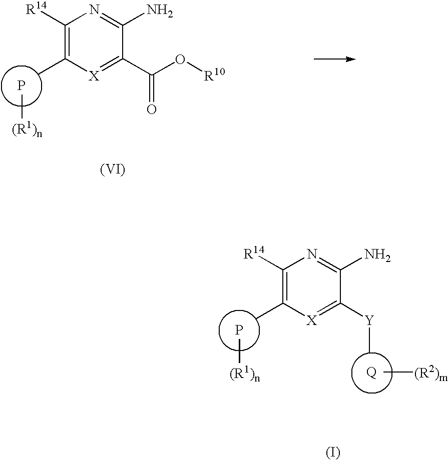

Thus, the amidation according to process B may be carried out by treating a compound of formula VI, wherein R¹⁰ is Cl-6alkyl, with an appropriate amine such as a compound of lo formula XI or 3-aminopyridine. The reaction can be performed neat or using a suitable solvent such as N,N-dimethylformamide, methylene chloride or ethyl acetate at a temperature ranging from −25° C. to +150° C. The reaction may be aided by using a base such as potassium carbonate, triethylanilne or 1,8-diazabicyclo[5.4.0]undec-7-ene or an acid such as trimethylaluminum or p-toulenesulfonic acid;

or,

the amidation of a compound of formula VI, wherein R¹⁰ is hydrogen, may be performed by activation of a compound of formula VI by treating the compound with coupling reagents such as 1-[3-(dimethylamino)propyl]-3-ethylcarbodiimide hydrochloride and 1-hydroxybenzotriazole hydrate, 1,3-dicyclohexylcarbodiimide and 1-hydroxybenzotriazole hydrate, 1,1′-carbonyldiimidazole or O-(7-azabenzotriazol-1-yl)-N,N,N′,N′-tetramethyluronium hexafluorophosphate or using an acyl halide reagent such as cyanuric chloride, oxalyl chloride, thionyl chloride or bromotrispyrrolidinophosphonium hexafluorophosphate followed by treatment with the appropriate amine such as a compound of formula XI or 3-aminopyridine.

C de-halogen coupling, of a compound of formula VIII with an appropriate aryl species to give a compound of formula I:

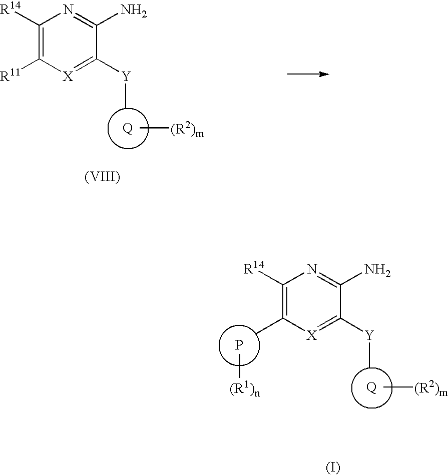

Thus, the de-halogen coupling according to process C may be carried out by using a suitable palladium catalyst such as Pd(PPh₃)₄, Pd(dppf)Cl₂ or Pd(OAc)₂ together with a suitable ligand such as P(tert-butyl)₃, 2-(dicyclohexylphosphino)biphenyl or a nickel catalyst such as nickel on charcoal or Ni(dppe)Cl₂ together with Zn and sodium triphenylphosphinetrimetasulfonate. A suitable base such as an alkyl amine e.g triethyl amine or potassium carbonate, sodium hydroxide or cesium fluoride may be used in the reaction, which is performed in the temperature range between +20° C. and +120.° C. in a suitable solvent such as toluene, tetrahydrofuran or N,N-dimethylformamide.

D amidation, wherein R² is a substituent that is not susceptible to certain agents in the reaction, of a compound of formula XII with the appropriate amine:

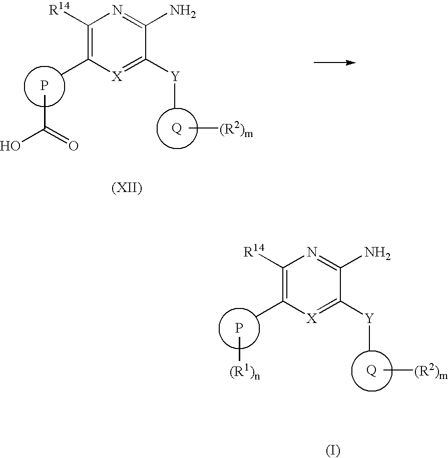

Thus, the amidation of a compound of formula XII according to process D may be performed by activation of the carboxylic acid function in a compound of formula XII, by treating the compound with coupling reagents such as 1-[3-(dimethylamino)propyl]-3-ethylcarbodiimide hydrochloride and 1-hydroxybenzotriazole hydrate, 1,3-dicyclohexylcarbodiimide and 1-hydroxybenzotriazole hydrate, 1,1′-carbonyldiimidazole or O-benzotriazol-1-yl-N,N′,N′-tetramethyluronium hexafluorophosphate or using an acyl halide reagent such as cyanuric chloride, oxalyl, chloride, thionyl chloride or bromotrispyrrolidinophosphonium hexafluorophosphate in a suitable solvent such as N,N-dimethylformamide, dioxane or tetrahydrofuran followed by treatment with the appropriate amine, HNR⁸R⁹ and at a reaction temperature between 25° C. and 70° C.

The hydrochloric salt of a compound of formula I may be obtained from a compound of formula I by treatment with hydrochloric acid at a temperature range between 0° C. and +25° C., in a suitable solvent such as methylene chloride, tetrahydrofuran or methylene chloride/methanol mixture.

### EXAMPLES

The invention will now be illustrated by the following non-limiting examples.

General Methods

All starting materials are commercially available or earlier described in the literature. The ¹H and ¹³C NMR spectra were recorded on Brucker 400 at 400 MHz and 100 MHz, respectively. The mass spectra were recorded utilising thermospray (Finnigan MAT SSQ 7000, buffer: 50 nM NH₄QAc in CH₃CN:H₂O; 3:7), electron impact (Finnigan MAT SSQ 710) or electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD) ionisation techniques.

### Example 1

### 3-Amino-6-bromo-N-pyridin-3-ylpyrazine-2-carboxamide

To 3-aminopyridine (10 g, 106 mmol) at 70° C. were added methyl 3-amino-6-bromo-2-pyrazinecarboxylate (1.0 g, 4.3 mmol; described in: Ellingson, R. C.; Henry, R. L. J. Am. Chem. Soc., 1949, 71, 2798-2800) and 1,8-diazabicyclo[5.4.0]undec-7-ene (645 μL, 4.3 mmol). The reaction solution was stirred for 4 h, diluted with water (75 mL) and extracted s with methylene chloride (3×50 mL). The combined organic layers were washed with a saturated ammonium chloride solution, dried (MgSO₄), filtered and evaporated in vacuo. The crude product was purified on a silica gel column using methylene chloride/ethanol, (9:1), as the eluent to give 750 mg (59% yield) of the title compound as a yellow solid: ¹H NMR (CDCl₃, 400 MHz) δ 9.50 (br s, 1 H), 8.82 (d, J=3 Hz, 1 H), 8.43 (dd, J=5, 2 Hz, 1 H), 8.31 (s, 1 H), 8.23 (ddd, J=8, 3 and 2 Hz, 1 H), 7.34 (dd, J=8, 5 Hz, 1 H); MS (TSP) m/z 294 (M⁺+1).

### Example 2

### 3-Amino-6-phenyl-N-pyridin-3-ylpyrazine-2-carboxamide

A mixture of 3-amino-6-bromo-N-pyridin-3-ylpyrazine-2-carboxamide (50 mg, 170 μmol), phenylboronic acid (31 mg, 255 μmol) and Pd(dppf)Cl₂×CH₂Cl₂, 1:1, (7 mg, 8.5 μmol) in toluene (5 mL), ethanol (0.35 mL) and a Na₂CO₃ solution (2 M, 0.35 mL) was stirred at 80° C. over night in a round bottom flask fitted with a condenser. Silica gel (0.5 g) was added to the reaction mixture and the mixture was concentrated to dryness. The residue was purified on a silica gel column using heptane/ethyl acetate, (1:1), as the eluent to give 51 mg (69% yield) of the title compound as a yellow solid: ¹H NMR (CDCl₃, 400 MHz) δ 9.96 (br s, 1 H), 8.82 (br s, 1 H), 8.72 (s, 1 H), 8.43 (br d, J=4 Hz, 1 H), 8.30 (d, J=8 Hz, 1 H), 7.92-7.89 (m, 2 H), 7.53 (t, J=7 Hz, 2 H), 7.46 (d, J=7 Hz, 1 H), 7.36 (dd, J=8, 5 Hz, 1 H); MS (TSP) m/z 292 (M⁺+1).

### Example 3

### 3-Amino-6-(2-methylphenyl)-N-pyridin-3-ylpyrazine-2-carboxamide

The compound was prepared as described in Example 2 using 2-methylphenylboronic acid: yield 42%; ¹H NMR (CDCl₃, 400 MHz) δ 9.87 (br s, 1 H), 8.76 (d, J=2 Hz, 1 H), 8.41 (s, 1 H), 8.40 (dd, J=5,1 Hz, 1 H), 8.26 (dd J=8; 2 Hz, 1 H) 7.43 (dd, J=6, 2 Hz, 1 H), 7.38-7.31 (m, 4 H), 2.45 (s, 3 H); MS (EI) m/z 305 (M⁺).

### Example 4

### 3-Amino-6-(4-cyanophenyl)-N-pyridin-3-ylpyrazine-2-carboxamide

The compound was prepared as described in Example 2 using 4-cyanophenylboronic acid: yield 36%; ¹H NMR (CDCl₃, 400 MHz) δ 9.80 (br s, 1 H), 8.83 (d, J=5 Hz, 1 H), 8.75 (s, 1 H), 8.45 (dd, J=5, 1 Hz, 1 H), 8.28 (ddd, J=8, 3 and 2 Hz, 1 H), 8.04-8.01 (m, 2 H), 7.83-7.80 (m, 2 H), 7.37 (dd, 8, 5 Hz, 1 H); MS (EI) m/z 316 (M⁺).

### Example 5

### 3-Amino-6-(3,4-methylenedioxyphenyl)-N-pyridin-3-ylpyrazine-2-carboxamide

The compound was prepared as described in Example 2 using 3,4-methylenedioxyphenylboronic acid: yield 63%; ¹H NMR (CDCl₃, 400 MHz) δ 9.90 (br s, 1 H), 8.82 (d, J=2 Hz, 1 H), 8.63, (s, 1 H), 8.42 (dd, J=5, 1 Hz, 1 H), 8.29 (ddd, J=8,2 and 2 Hz, 1 H), 7.77-7.71 (m, 1 H), 7.54-7.52 (m, 1 H), 7.39-7.36 (m, 2 H), 7.35 (dd, J=8, 5 Hz, 1 H), 6.95 (d, J=8 Hz, 1 H), 6.05 (s, 2 H); MS (EI) m/z 335 (M⁺).

### Example 6

### 3-Amino-6-(2-thienyl)-N-pyridin-3-ylpyrazine-2-carboxamide

The compound was prepared as described in Example 2 using 2-thienylboronic acid: yield 57%; mp 178-189° C., ¹H NMR (CDCl₃, 400 MHz) δ 9.85 (br s, 1 H), 8.81 (d, J=2 Hz, 1 H), 8.66 (s, 1 H), 8.42 (dd, J=5, 1 Hz, 1 H), 8.31-8.28 (m, 1 H), 7.52 (d, J=4 Hz, 1 H), 7.40 (d, J=5 Hz, 1 H), 7.35 (dd, J=8, 5 Hz, 1 H), 7.14 (dd, J=5, 4 Hz, 1 H); MS (EI) m/z 297 (M⁺).

### Example 7

### 3-Amino-6-(3-nitrophenyl)-N-pyridin-3-ylpyrazine-2-carboxamide

The compound was prepared as described in Example 2 using 3-nitrophenylboronic acid: yield 42%; ¹H NMR (CDCl₃, 400 MHz) δ 9.84 (br s, 1 H), 8.94 (br s, 1 H), 8.79 (s, 1 H), 8.77 (t, J=2 Hz, 1 H), 8.47 (d,J=4 Hz, 1 H), 8.31-8.25 (m,2 H), 8.23 (dd, J=9, 1 Hz, 1 H), 7.71 (t, J=8 Hz, 1 H), 7.39 (dd, J=8, 5 Hz, 1 H); MS (EI) m/z 336 (M⁺).

### Example 8

### 3-Amino-6-(3,5-bistriflouromethylphenyl)-N-pyridin-3-ylpyrazine-2-carboxamide

The compound was prepared as described in Example 2 using 3,5-bistrifluoromethylphenylboronic acid: yield 44%; mp 220-222° C.; ¹H NMR (CDCl₃, 400 MHz) δ 9.77 (br s, 1 H), 8.84 (b, J=2 Hz, 1 H), 8.77 (s, 1 H), 8.45 (dd, J=5, 1 H 8.32(s, 2 H), 8.26 (ddd, J=8, 3 and 2 Hz, 1 H), 7.94 (s, 1 H), 7.38 (dd, J=8, 5 Hz, 1 H); MS (EI) m/z 427 (M⁺).

### Example 9

### 3-Amino-6-(3-thienyl)-N-pyridin-3-ylpyrazine-2-carboxamide

The compound was prepared as described in Example 2 using 3-thienylboronic acid: yield to 23%; MS (EI) m/z 297 (M⁺).

### Example 10

### 3-Amino-6-(4-fluorophenyl)-N-pyridin-3-ylpyrazine-2-carboxamide

The compound was prepared as described in Example 2 using 4-fluorophenylboronic acid: yield 48%; mp 193-197° C.; ¹H NMR (CDCl₃, 400 MHz) δ 9.90 (br s, 1 H), 8.81 (d, J=2 Hz, 1 H), 8.67(s, 1 H), 8.43 (dd, J=5, 1 Hz, 1 H), 8.30 (ddd, J=8, 2 and 2 Hz, 1 H), 7.89-7.86 (m, 2 H), 7.36 (dd, J=8, 5 Hz, 1 H), 7.22 (t, J=9 Hz, 2 H), MS (EI) m/z 309 (M⁺).

### Example 11

### 3-Amino-6-(4-chlorophenyl)-N-pyridin-3-ylpyrazine-2-carboxamide

The compound was prepared as described in Example 2 using 4-chlorophenylboronic acid: yield 48%; ¹H NMR (CDCl₃, 400 MHz) δ 9.88 (br s, 1 H), 8.82 (d, J=2 Hz, 1 H), 8.69 (s, 1 H), 8.43 (dd, J=5, 1 Hz, 1 H), 8.29 (ddd, J=8, 2 and 1 Hz, 1 H), 7.86-7.82 (m, 2 H) 7.54-7.44 (m, 2 H), 7.36 (dd, J=8, 5 Hz, 1 H); MS (EI) m/z 325 (M⁺).

### Example 12

### 3-Amino-6-(2,3-dichlorophenyl)-N-pyridin-3-ylpyrazine-2-carboxamide

The compound was prepared as described in Example 2 using 2,3-dichlorophenylboronic acid: yield 75%; mp 239-241° C.; ¹H NMR (DMSO-d6, 400 MHz) δ 10.52 (s, 1 H), 8.94 (d, J=2 Hz, 1 H), 8.62 (s, 1 H), 8.33-8.32 (m, 1 H), 8.18 (d, J=8 Hz, 1 H), 7.82 (br s, 2 H), 7.76-7.73 (m, 2 H), 7.51 (t, J=8 Hz, 1 H), 7.40 (dd, J=8, 5 Hz, 1 H); ¹³C NMR (DMSO-d6, 100 MHz) δ 164.89, 154.28, 148.02, 144.95, 142.70, 137.93, 137.71, 134.66, 132.30, 130.67, 130.47, 129.90, 128.43, 128.05, 123.74, 123.46; MS (EI) m/z 360 (M⁺).

### Example 13

### 3-Amino-6-(2,4-dichlorophenyl)-N-pyridin-3-ylpyrazine-2-carboxamide

The compound was prepared as described in Example 2 using 2,4-dichlorophenylboronic acid: yield 41%; ¹H NMR (DMSO-d6, 400 MHz) δ 10.52 (s, 1 H), 8.94 (br s, 1 H), 8.64 (s, 1 H), 8.34 (d, J=4 Hz, 1 H), 8.18 (br d, J=8 Hz, 1 H), 7.88 (d, J=8 Hz, 1 H), 7.81 (br s, 2 H), 7.79 (d, J=2 Hz, 1 H), 7.69 (dd, J=8, 2 Hz, 1 H), 7.41 (dd, J=8, 4 Hz, 1 H); ¹³C NMR (DMSO-d6, 100 MHz) δ 164.88, 154.20, 147.97, 144.97, 142.64, 136.81, 134.66, 134.39, 133.71, 133.32, 132.32, 129.32, 127.99, 127.76, 123.98, 123.50; MS (EI) m/z 360 (M⁺).

### Example 14

### 3-Amino-6-(2,4-difluorophenyl)-N-pyridin-3-ylpyrazine-2-carboxamide

The compound was prepared as described in Example 2 using 2,4-difluorophenylboronic acid: yield 31%; mp 232-234° C.; ¹H NMR (DMSO-d6, 400 Hz) δ 10.57 (s, 1 H), 8.96 (d, J=2 Hz, 1 H), 8.68 (d, J=3 Hz, 1 H), 8.36-8.30 (m, 2 H), 8.21-8.18 (m, 1 H), 7.78 (br s, 2 H), 7.48-7.39 (m, 2 H), 7.27 (dt, J=8, 2 Hz, 1 H); MS (ES) m/z 328 (M⁺+1).

### Example 15

### 3-Amino-6-(3,4-difluorophenyl)-N-pyridin-3-ylpyrazine-2-carboxamide

The compound was prepared as described in Example 2 using 3,4-difluorophenylboronic acid: yield 66%; mp 232-234° C.; ¹H NMR (DMSO-d6, 400 MHz) δ 10.63 (s, 1 H), 8.98 (s, 1 H), 8.98 (s, 1 H), 8.96 (d, J=2 Hz, 1 H), 8.46 (ddd, J=13, 8 and 2 Hz, 1 H), 8.37 (dd, J=5, 1 Hz, 1 H), 8.20-8.17 (m, 1 H), 8.11-8.04 (m, 1 H), 7.78 (br s, 2 H), 7.54 (dt, J=10, 9 Hz, 1 H), 7.45 (dd, J=8,5 Hz, 1 H); MS (ES) m/z 328 (M⁺+1).

### Example 16

### 3-Amino-6-(3-chloro-4-fluorophenyl)-N-pyridin-3-ylpyrazine-2-carboxamide

The compound was prepared as described in Example 2 using 3-chloro-4-fluorophenylboronic acid: yield 44%; mp 236-238.5° C.; ¹H NMR (DMSO-d6, 400 MHz) δ 10.66 (s, 1 H), 8.99 (s, 1 H), 8.97 (d, J=2 Hz, 1 H), 8.54 (dd, J7, 2 Hz, 1 H), 8.37 (d, J=4Hz, 1 H), 8.27 (ddd, J=9, 5 and 2 Hz, 1 H), 8.20 (dd, J=8, 2 Hz, 1 H), 7.83-7.64 (m, 2 H), 7.53 (t, J=9 Hz, 1 H), 7.44 (dd, J=8, 5 Hz, 1 H); MS (ES) m/z 344 (M⁺+1).

### Example 17

### 3-Amino-6-[4-fluoro-3-methylphenyl]-N-pyridin-3-ylpyrazine-2-carboxamide

The compound was prepared as described in Example 2 using 4-fluoro-3-methylphenylboronic acid: yield 76%; mp 186-189° C.; ¹H NMR (DMSO-d6, 400 MHz) δ 10.57 (s, 1 H), 8.99 (d, J=2 Hz, 1 H), 8.92 (s, 1 H), 8.36 (dd, J=5, 1 Hz, 1 H), 8.22 (ddd, J=8, 2 and 2 Hz, 1 H), 8.18 (dd, J=8, 2 Hz, 1 H), 8.13-8.09 (m, 1 H), 7.67 (br s, 2 H), 7.44 (dd, J=8, 5 Hz, 1 H), 7.24 (t, J=9 Hz, 1 H), 2.34 (d, J=1 Hz, 3 H); MS (ES) m/z 324 (M⁺+1).

### Example 18

### 3-Amino-6-(3,4-dimethylphenyl)-N-pyridin-3-ylpyrazine-2-carboxamide

The compound was prepared as described in Example 2 using 3,4-dimethylphenylboronic acid: yield 80%; mp 178-182° C.; ¹H NMR (DMSO-d6, 400 MHz) δ 10.56 (s, 1 H), 8.99 (d, J=2 Hz, 1 H), 8.90 (s, 1 H), 8.36 (dd, J=5, 1 Hz, 1 H), 8.24-8.21 (m, 1 H), 8.01 (s, 1 H), 7.95 (dd, J=8, 2 Hz, 1 H),7.63 (br s, 2 H), 7.44 (dd, J=8, 5 Hz, 1 H), 7.25 (d, J=8 Hz, 1 H), 2.33 (s, 3 H), 2.28 (s, 3 H); MS (ES) m/z 320 (M⁺+1).

### Example 19

### 3-Amino-6-(3-fluorophenyl)-N-pyridin-3-ylpyrazine-2-carboxamide

The compound was prepared as described in Example 2 using 3-fluorophenylboronic acid: yield 92%; mp 234.5-238° C.; ¹H NMR (DMSO-d6, 400 MHz) δ 10.63 (s, 1 H), 8.99 (s, 1 H), 8.97 (d, J=2 Hz, 1 H), 8.37 (dd, J=5, 1 Hz, 1 H), 8.22-8.18 (m, 2 H), 8.07 (d, J=8 Hz, 1 H), 7.78 (br s, 2 H), 7.52 (dt, J=8, 6 Hz, 1 H), 7.44 (dd, J=8, 5 Hz, 1 H), 7.22 (dt, J=8, 2 Hz, 1 H); MS (ES) ) m/z 310 (M⁺+1).

### Example 20

### 3-Amino-6-(2-fluorophenyl)-N-pyridin-3-ylpyrazine-2-carboxamide

The compound was prepared as described in Example 2 using 2-fluorophenylboronic acid: yield 82%; mp 221-225° C.; ¹H NMR (DMSO-d6, 400 MHz) δ 10.56(s, 1 H), 8.97 (d, J=2 Hz, 1 H), 8.71 (d, J=3 Hz, 1 H), 8.35 (dd, J=5, 1 Hz, 1 H), 8.25 (dt, J=8, 2 Hz, 1 H) 8.22-8.19 (m, 1 H), 7.77 (br s, 2 H), 7.50-7.41 (m, 2 H), 7.39-7.33 (m, 2 H); MS (ES) m/z 310 (M⁺+1).

### Example 21

### 3-Amino-6-bromo-5-methyl-N-pyridin-3-ylpyrazine-2-carboxamide

Trimethyl aluminum (2.0 M in hexane, 2.0 mL, 4.0 mmol) was added dropwise to a stirred solution of methyl 3-amino-6-bromo-5-methylpyrazine-2-carboxylate (0.49 g, 2.0 mmol; described in: Bicking, J. B. J. Med. Chem, 1967, 10, 598-602) and 3-aminopyridine in methylene chloride (12 mL) under an atmosphere of nitrogen. The resulting mixture was stirred at room temperature for 1.5 h and at reflux for 27 h. After cooling to room temperature, water was added and stirring was continued for another 10 min. The aqueous is phase was extracted with methylene chloride and the combined organic phases were washed with water, dried (MgSO₄), and the solvent was evaporated. The crude product was purified by column chromatography using methylene chloride/methanol, (95:5), to give 0.48 g (77% yield) of the title compound: ¹H NMR (DMSO-d6, 400 MHz) δ 10.44 (s, 1 H), 8.96 (s, 1 H), 8.33 (m, 1 H), 8.17 (m, 1 H), 7.67 (br s, 2 H), 7.38 (m, 1 H), 2.48 (s, 3 H); ¹³C NMR (DMSO-d6) δ 168.3, 161.3, 158.0, 148.9, 146.8, 138.8, 132.1, 127.4, 126.6, 126.4, 27.7.

### Example 22

### tert-Butyl 4-(2-hydroxyethyl)pyridin-3-ylcarbamate

tert-Butyl pyridin-3-ylcarbamate (2 g, 10.3 mmol; described in: Kelly, T. A.; McNiel, D. W., Tetrahedron Lett. 1994, 35, 9003-9006) was dissolved under inert gas atmosphere in tetrahydrofuran (60 mL) and the solution was cooled to −78° C. tert-Butyl lithium (14 mL, 1.7 M in pentane) was added dropwise and stirring was continued for 3 h. Ethylene oxide (1 mL, 20 mmol) was added dropwise and the reaction was allowed to warm up to room temperature. Saturated ammonium chloride solution was added (5 mL). The organic layer was separated and dried over magnesium sulfate. Filtration and removal of the solvent in vacuo yielded a residue which was purified by column chromatography on silica gel using heptane/ethyl acetate, (10:1→0:100), as the eluent to give 1.7 g (70% yield) of the title compound as a white solid: ¹H NMR (CD₃OD, 400 MHz) δ8.66 (s, 1 H), 8.22 (d, J=5 Hz, 1 H), 7.33 (d, J=5 Hz, 1 H), 3.83 (t, J=6 Hz, 2 H), 2.89 (t, J=7 Hz, 2 H), 1.54 (s, 9 H); MS (ES) m/z 239 (M⁺+1).

### Example 23

### tert-Butyl 4-(2-pyrrolidin-1-ylethyl)pyridin-3-ylcarbamate

tert-Butyl 4-(2-hydroxyethyl)pyridin-3-ylcarbamate (1 g, 4.2 mmol) was dissolved in methylene chloride (40 mL) under inert gas atmosphere and cooled to 0° C. Methanesulfonyl chloride (0.48 mL, 6.3 mmol) and triethylamine.(1.8 mL, 12.6 mmol) were added and stirring was continued for 1.5 h. Pyrrolidine (1.76 mL, 21 mmol) was added and the reaction mixture was stirred for 12 h at room temperature. Saturated aqueous sodium chloride solution (5 mL) was added and the organic layer was separated and dried over sodium sulfate. Filtration and removal of the solvent in vacuo yielded a residue, which was purified by chromatography on silica gel using ethyl acetate/heptane, (1:8→1:1), as the eluent to give 730 mg (60% yield) of the title compound as an oil: ¹ H NMR (CDCl₃, 400 MHz) δ 9.09 (br s, 1 H), 8.18 (d, =5 Hz, 1 H), 6.96 (d, J=5 Hz, 1 H), 2.76 (m, 4 H), 2.66 (m,4 H), 1.89 (m, 4 H), 1.54 (s, 9 H); MS (ES) m/z ²⁹² (M⁺+1).

### Example 24

### 4-(2-Pyrrolidin-1-ylethyl)pyridin-3-amine

tert-Butyl 4-(2-pyrrolidin-1-ylethyl)pyridin-3-ylcarbamate (0.8 g, 2.8 mmol) was dissolved in methylene chloride (20 mL). Trifluoroacetic acid (1.05 mL, 14 mmol) was added and stirring was continued for 30 min. The solvent was removed in vacuo and ethyl acetate (5 mL) were added and removed in vacuo. This procedure was repeated 3 times. The residue was dissolved in methanol (50 mL) and DOWEX-OH was added until the methanolic solution was basic. Filtration and removal of the solvent in vacuo gave the title compound in 80% yield: ¹H NMR (CD₃OD, 400 MHz) δ 7.95 (s, 1 H), 7.75 (d, J=5 Hz, 1 H), 7.04 (d, J=5 Hz, 1 H), 2.75 (m. 4 H), 2.66 (m, 4 H), 1.86 (m, 4 H); MS (ES) m/z 192 (M⁺+1).

### Example 25

### 3-Amino-6-bromo-N-[4-(2-pyrrolidin-1-ylethyl)pyridin-3-yl]pyrazine-2-carboxamide

3-Amino-6-bromopyrazine-2-carboxylic acid (148 mg, 0.68 mmol; described in: Ellingson, R. C.; Henry, R. L., J. Am. Chem. Soc. 1949, 2798-2800), 4-(2-pyrrolidin-1-ylethyl)pyridin-3-amine (107 mg, 0.56 mmol), 2-(1H-benzotriazol-1-yl)-1,1,3,3-tetramethyluroniumtetrafluoroborate (276 mg, 0.87 mmol), 1-hydroxybenzotriazole hydrate (114 mg, 0.86 mmol) and N,N-diisopropylethylamine (0.2 mL, 1.15 mmol) were suspended in 8 mL acetonitrile and stirred under inert gas atmosphere at room temperature for 12 h. The solvent was removed in vacuo and the residue was separated between methylene chloride and saturated aqueous sodium hydrogen carbonate solution. The organic layer was dried over sodium sulfate. Filtration and removal of solvent in vacuo yielded the crude product which was purified by chromatography on silica using a gradient ethyl acetate/methanol, (10:1), to ethyl acetate/methanol/triethyl amine (4:1:0.05) as an eluent to give 200 mg (91% yield) of the title compound as a brown oil: ¹H NMR (DMSO-d6, 400MHz) δ 10.51 (brs, 1 H),8.68 (s, 1 H), 8.43, s (1 H), 8.33 (d, J=5 Hz, 1 H), 7.72 (br s, 2 H), 7.35 (d, J=5 Hz, 1 H), 2.77 (m, 2 H), 2.67 (m, 2 H), 2.49 (m, 4 H), 1.63 (m, 4 H).

### Example 26

### 3-Amino-6-(4-chlorophenyl)-5-methyl-N-pyridin-3-ylpyrazine-Z-carboxamide

3-Amino-6-bromo-5-methyl-N-pyridin-3-ylpyrazine-2-carboxamide (0.290 g, 0.94 mmol), 4-chlorophenylboronic acid (0.161 g, 1.0 mmol), and Pd(dppf)Cl₂×CH₂Cl₂ (0.038 g, 0.047 mmol), were mixed in toluene/ethanol, (15:3 mL), and a saturated Na₂CO₃ (aq) solution (1.5 mL). Nitrogen gas was bubbled through the reaction mixture for 5 min and the mixture was heated for 16 h. Silica gel was added and the solvent was evaporated. Purification by column chromatography using methylene chloride/methanol, (95:5), gave 0.318 g (99% yield) of the title compound: ¹H NMR (DMSO-d6, 400 MHz) δ 10.39 (s, 1 H), 8.94 (d, J=2 Hz, 1 H), 8.33 (dd, J=5, 1 Hz, 1 H), 8.19 (m, 1 H), 7.79 (m, 2 H), 7.56 (br s, 2H), 7.55 (m, 2 H), 7.40 (dd, J=8,5 Hz, 1 H), 2.50 (s, 3 H); ¹³C NMR (DMSO-d6, 100 MHz) δ 165.1, 154.8, 153.5, 144.8, 142.7, 138.7, 136.9, 143.7, 132.6, 131.1, 128.1, 128.0, 123.4, 121.7, 23.1; MS (TSP) m/z 340 (M⁺+1)

### Example 27

### 3-Amino-6-(2,4-dichlorophenyl)-N-[4-(2-pyrrolidin-1-ylethyl)pyridin-3-yl]pyrazine-2-carboxamide

2,4-Dichlorobenzeneboronic acid (0.029 g, 0.15 mmol), 3-amino-6-bromo-N-[4-(2-pyrrolidin-1-ylethyl)pyridin-3-yl]pyrazine-2-carboxamide (0.03 g, 0.077 mmol), Na₂CO₃ (0.025 g, 0.24 mmol), and Pd(dppf)Cl₂×CH₂Cl₂ (3 mg, 0.004 mmol) were suspended in ethylene glycol dimethyl ether/water, (2.5:0.6 mL), and heated in a microwave oven at 160° C. for 10 min. Silica was added and the solvent was evaporated. Purification by column chromatography on silica using methylene chloride/methanol, (95:5), as the eluent gave 0.020 g of the title compound as a yellow solid: MS (TSP) m/z 457 (M⁺+1).

### Example 28

### 3-Amino-6-(3-chloro-4-fluorophenyl)-N-[4-(2-pyrrolidin-1-ylethyl)pyridin-3-yl]pyrazine-2-carboxamide

The title compound was prepared as described for Example 27 using 3-chloro-4-fluoro-benzeneboronic acid and 3-amino-6-bromo-N-[4-(2-pyrrolidin-1-ylethyl)pyridin-3-yl]pyrazine-2-carboxamide: yield 95%; MS (TSP) m/z 441 (M⁺+1).

### Example 29

### 3-Amino-6-(2,4-dichlorophenyl)-N-[4-(2-pyrrolidin-1-ylethyl)pyridin-3-yl]pyrazine-2-carboxamide hydrochloride

HCl in diethyl ether (1.0 M, 0.20 mmol) was added to a solution of 3-amino-6-(2,4-dichlorophenyl)-N-(4-(2-pyrrolidin-1-ylethyl)pyridin-3-yl]pyrazine-2-carboxamide (0.020 g, 0.044 mmol) in methylene chloride (7 mL). The resulting mixture was stirred at room temperature for 30 min and the precipitate was filtered off, washed with diethyl ether and dried in vacuo to give 0.020 g (86% yield) of the title compound: ¹H NMR (D₂O, 400 MHz) 69.19 (s, 1 H), 8.54 (s, 1 H), 8.53 (d, J=6 Hz, 1 H), 7.87 (d, J=6 Hz 1 H), 7.65 (d, J=2Hz, 1 H),7.60(d, J=8 Hz, 1 H), 7.47 (dd, J=8,2 Hz, 1 H), 3.50 (m, 4 H), 3.29 (m, 2 H), 2.90 (m, 2 H), 1.85 (m, 4 H); MS (TSP) m/z 457 (M⁺+1).

### Example 30

### 3-Amino-6-(3-chloro4-fluorophenyl)-N-[4-(2-pyrrolidin-1-ylethyl)pyridin-3-yl]pyrazine-2-carboxamide hydrochloride

The title compound was prepared as described for Example 29 using: 3-amino-6-(3-chloro-4-fluorophenyl)-N-[4-(2-pyrrolidin-1-ylethyl)pyridin-3-yl]pyrazine-2-carboxamide: yield 90%; ¹H NMR (DMSO-d6) δ 11.06 (br s, 1 H), 10.74 (s, 1 H), 9.02 (s, 1 H), 8.94 (s, 1 H), 8.69 (d, J=5 Hz, 1 H), 8.57 (dd, J=7, 2 Hz, 1 H), 8.28 (m, 1 H), 7.86 (d, J=5 Hz, 1 H), 7.53 (t, J=9 Hz, 1 H), 3.49 (m, 4 H), 3.28 (m, 2 H), 2.99 (m, 2 H), 1.83 (m, 4 H); MS (TSP) m/z 441 (M⁺+1).

### Example 31

### 3-Amino-6-(2-furyl)-N-pyridin-3-ylpyrazine-2-carboxamide

2-Furylbotonic acid (62 mg, 0.55 mmol) and 3-amino-6-bromo-N-pyridin-3-ylpyrazine-2-carboxamide (88 mg, 0.29 mmol) were suspended under inert gas atmosphere in tetrahydrofuran (4 mL). Sodium carbonate (2 mL, 2 M in water, 4 mmol) Pd(dppf)Cl₂×CH₂Cl₂ (20 mg, 0.02 mmol) were added and the reaction mixture was vigorously stirred at 40° C. for 1 h. Water (5 mL) and ethyl acetate (15 mL) were added and the layers were separated. The aqueous layer was extracted with ethyl acetate and the combined organic layers were dried over magnesium sulfate. Filtration and removal of the solvent in vacuo yielded a residue which was purified by column chromatography on silica using a gradient ethyl acetate/heptane, (1:1), to ethyl acetate methanol, (10:1), as eluent to give 65 mg (42% yield) of the title compound as a solid: ¹H NMR (DMSO-d6, 400 MHz) δ 10.53 (s, 1 H), 8.99 (m, 1 H), 8.67 (s, 1H), 8.36 (m, J=4 Hz, 1 H), 8.24 (m, J=8 Hz, 1 H), 7.80 (dd, J=2, 1 Hz, 1 H), 7.72 (br s, 2 H), 7.44 (dd, J=8, 5 Hz, 1 H), 7.35 (dd, J=4, 1 Hz, 1 H), 6.91. (dd, J=4 Hz, 2 Hz, 1 H); ¹³C NMR (DMSO-d6, 100 MHz) δ 165.1, 154.2, 151.3, 145.2, 143.6, 143.3, 143.0, 135.0, 132.5, 128.8, 123.9, 123.7, 112.4, 108.1; MS (ES) m/z 282.03 (M⁺+1).

### Example 32

### 3-Amino-6-(4-hydroxyphenyl)-N-pyridin-3-ylpyrazine-2-carboxamide

4-(4,4,5,5-Tetramethyl-1,3,2-dioxaborolan-2-yl)phenol (0.20 g, 0.91 mmol), 3-amino-6-bromo-N-(3-pyridinyl)-2-pyrazinecarboxamide (0.267 g, 0.91 mmol), Na₂CO₃ (0.291 g, 2.75 mmol), and Pd(dppf)Cl₂×CH₂Cl₂ (0.037 g, 0.045 mmol) were suspended in dimetoxymethane/water (3:1. mL) and heated in a microwave oven at 160° C. for 10 min. Silica was added and the solvent evaporated. Purification by column chromatography on silica using methylene chloride/methanol, (95:5), as the eluent and subsequent wash with methylene chloride gave 0.102 g (18% yield) of the title compound as a yellow solid: ¹H NMR (DMSO-d6, 400 MHz) δ 10.51 (s, 1 H), 9.66 (s, 1 H), 8.98 (d, J=3 Hz, 1 H), 8.83 (s, 1H), 8.36 (m, 1 H), 8.21 (m, 1 H), 8.06 (m, 2 H), 7.52 (br s, 2 H), 7.42 (dd, J=8, 5 Hz, 1 H), 6.86 (m, 2 H); ¹³CNMR (DMSO-d6, 100 MHz) δ 165.2, 157.8, 153.6, 144.9, 144.2, 142.8, 139.3, 134.6, 128.2, 127.2, 126.7, 123.4, 122.9, 115.4; MS (ES) m/z 308 (M⁺+1).

### Example 33

### 3-Amino-6-(4-hydroxyphenyl)-N-pyridin-3-ylpyrazine-2-carboxamide-hydrochloride

HCl in diethyl ether (1 M, 1.2 mL) was added to a stirred solution of 3-amino-6-(4-hydroxyphenyl)-N-pyridin-3-ylpyrazine-2-carboxamide (0.090 g, 0.29 mmol) in methylene chloride/methanol, (10:10 mL). The resulting mixture was stirred at room temperature for 15 min and the solvent was evaporated to give 0.097 g (yield 87%) of the title compound as a yellow solid: ¹H NMR (DMSO-d6, 400 MHz) δ 10.87 (s, 1 H), 9.71 (br s, 1 H), 9.26 (d, J=2 Hz, 1 H), 8.87 (s, 1 H), 8.68 (d, J=9 Hz, 1 H), 8.57 (d, J=5 Hz, 1 H), 8.07 (m, 2 H), 7.86 (dd, J=9, 5 Hz, 1 H), 5.57 (br s, 1 H), 6.88 (m, 2 H); MS (ES) m/z 308 (M⁺+1).

### Example 34

### 3-Amino-6-[4-(aminocarbonyl)phenyl]-N-pyridin-3-ylpyrazine-2-carboxamide

Triethyl amine (33.2 mg, 0.255 mmol) in N,N-dimethylformamide (0.10 mL) was added to a solution of 4-(5-amino-6-[(pyridin-3-ylamino)carbonyl]pyrazin-2-yl]benzoic acid (52.9 mg, 0.150 mmol) and O-benzotriazol-1-yl-N,N,N′,N′-tetramethyluronium hexafluorophosphate (0.18 mmol) in N,N-dimethylformamide (8.5 mL ). NH₃ (2.33 mg, 0.15 mmol) in dioxane (0.33 mL) was added and the mixture was shaken at room temperature for 24 h. Most of the solvent was removed and the crude reaction mixture was dissolved in dimethyl sulfoxide (1 mL) and purified by chromatography with 30 acetonitrile/water (5:95, increasing to, 95:5, for 12 minutes, XTerra C8-column 19×100 mm). The product was further purified by a second chromatography with acetonitrile/water (10:90 increasing to 60:10 in 13 minutes, XTerra C8-column 19×300 mm) to give 2.7 mg (5% yield) of the title compound: MS (ES) m/z 335 (M⁺+1).

### Example 35

### 4-{5-Amino-6-[(pyridin-3-ylamino)carbonyl]pyrazin-2-yl}benzoic acid

Pd(PPh₃)₄ (1.05 g, 0.91 mmol) was added to a to a solution of 3-amino-6-bromo-N-pyridin-3-ylpyrazine-2-carboxamide (2.0 g, 6.8 mmol); 4-carboxyphenylboronic acid (1.12 g, 6.7 mmol), and sodium carbonate (2.88 g, 27.2 mmol) in tetrahydrofuran/water, (1:1, 240 mL), and the resulting mixture was heated at 75° C. for 16 days. The solvent was evaporated and the residue dissolved in water. The aqueous phase was extracted with ethyl acetate and then neutralized (pH 7) using HCl (10%, aq). The formed crystals were filtered off and dried in vacuo to give 1.7 g (77% yield) of the title compound: MS (ES) m/z 336 (M⁺+1).

Pharmaceutical Formulations

According to one aspect of the present invention there is provided a pharmaceutical formulation comprising a compound of formula I, as a free base or a pharmaceutically acceptable salt thereof, for use in the prevention and/or treatment of conditions associated with glycogen synthase kinase-3.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream or for rectal administration as a suppository.

In general the above compositions may be prepared in a conventional manner using conventional excipients, pharmaceutical diluents or inert carriers.

Suitable daily doses of the compounds of formula I in the treatment of a mammal, including man are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

The following illustrate representative pharmaceutical dosage forms containing a compound of formula I, as a free base or a pharmaceutically acceptable salt thereof (hereafter compound X), for preventive or therapeutic use in mammals:

| (a): Tablet | Mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |

| (b): Capsule | Mg/capsule |
|---|---|
| Compound X | 10 |
| Lactose | 488.5 |
| Magnesium stearate | 1.5 |

| (c): Injection | (50 mg/ml) |
|---|---|
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | up to 100% |

The above formulations may be obtained by conventional procedures well known in the pharmaceutical art.

Medical Use

Surprisingly, it has been found that the compounds defined in the present invention, as a free base or a pharmaceutically acceptable salt thereof, are well suited for inhibiting glycogen synthase kinase-3 (GSK3). Accordingly, the compounds of the present invention are expected to be useful in the prevention and/or treatment of conditions associated with glycogen synthase kinase-3 activity, i.e. the compounds may be used to produce an inhibitory effect of GSK3 in mammals, including man in need of such prevention and/or treatment.

GSK3 is highly expressed in the central and peripheral nervous system and in other tissues. Thus, it is expected that the compounds of the invention are well suited for the prevention and/or treatment of conditions associated with glycogen synthase kinase-3 in the central and peripheral nervous system. In particular, such compounds of the invention are expected to be suitable for prevention and/or treatment of conditions associated with especially, dementia, Alzheimer's Disease, Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, Type I and Type II diabetes and diabetic neuropathy, hair loss and contraceptive medication.

The dose required for the therapeutic or preventive treatment of a particular disease will necessarily be varied depending on the host treated, the route of administration and the severity of the illness being treated.

The present invention relates also to the use of a compound of formula I as defined hereinbefore, in the manufacture of a medicament for the prevention and/or treatment of conditions associated with GSK3.

In the context of the present specification, the term “therapy” includes treatment as well as prevention, unless there are specific indications to the contrary. The terms “therapeutic” and “therapeutically” should be construed accordingly.

The invention also provides a method of treatment and/or prevention of conditions associated with GSK3, in a patient suffering from, or at risk of, said condition, which comprises administering to the patient an effective amount of a compound of formula I, as hereinbefore defined.

Non-Medical Use

In addition to their use in therapeutic medicine, the compounds of formula I as a free base or a pharmaceutically acceptable salt thereof, are also useful as pharmacological tools in the development and standardisation of in vitro and in vivo test systems for the evaluation of the effects of inhibitors of GSK3 related activity in laboratory animals such as cats, dogs, rabbits, monkeys, rats and mice, as part of the search for new therapeutics agents.

Pharmacology

Determination of ATP Competition in Scintillation Proximity GSK3β Assay.

GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, US), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi [γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 μM.

The following abbreviations have been used:

MOPS Morpholinepropanesulfonic acid

EDTA Ethylenediaminetetraacetic acid

BSA Bovin Serum Albumin

ATP Adenosine Triphophatase

SPA Scintillation Proximity Assay

GSK3 Glycogen Synthase Kinase 3

Pd(dppf)Cl₂ [1.1′-Bis(diphenylphosphino)ferrocene]dichloropalladium(II)

Ni(dppe)Cl₂ [1.1′-Bis(diphenylphosphino)ethane]dichloronickel(II).

Results

Typical Kᵢ values for the compounds of the present invention are in the range of about 0.001 to about 10,000 nM, preferably about 0.001 to about 1000 nM, particularly preferred about 0.001 nM to about 300 nM.

1. A compound of formula I 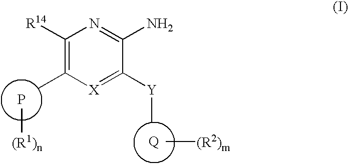
wherein:
Y is CONR³, NR³CO, SO₂NR³, NR³SO₂, CH₂NR³, NR³CH₂, NR³CONR³, C₁₋₆alkylene, CH₂CO, COCH₂, CH═CH, OCH₂ or CH₂O;
X is CH or N;
P is phenyl or a 5 or 6 membered heteroaromatic ring containing one or more heteroatoms selected from N, O or S and said phenyl ring or 5 or 6 membered heteroaromatic ring may optionally be fused with a 5 or 6 membered saturated, partially saturated or unsaturated ring containing atoms selected from C, N, O or S;
Q is phenyl or a 5 or 6 membered heteroaromatic ring containing one or more heteroatoms selected from N, O or S wherein at least one atom is nitrogen;
R¹ is halo, nitro, C₀₋₆alkylCN, C₀₋₆alkylOR⁸, fluoromethyl, difluoromethyl, trifluoromethyl, C₀₋₆alkylNR⁸R⁹, C₀₋₆alkylCONR⁸R⁹, C₀₋₆alkylNR⁸(CO)R⁹, NR⁸(CO)OR⁹, C₀₋₆alkylO(CO)R⁸, C₀₋₆alkylSO₂R⁸, C₀₋₆alkylSOR³, C₀₋₆alkylCOR⁸, C₀₋₆alkylO(CO)OR⁸, C₁₋₆alkylCO₂R⁸, OC₀₋₆alkylSO₂R⁸, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted by one or more A;
R² is halo, nitro, CHO, C₀₋₆alkylCN, OC₁₋₆alkylCN, C₀₋₆alkylOR⁴, OC₁₋₆alkylOR⁴, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R⁵, OC₁₋₆alkylOC₁₋₆alkylNR⁴R⁵, NR⁴OR⁵ C₀₋₆alkylCO₂R⁴, OC₁₋₆alkylCO₂R⁴, C₀₋₆alkylCONR⁴R⁵, OC₁₋₆alkylCONR⁴R⁵, OC₁₋₆alkylNR⁴(CO)R⁵, C₀₋₆alkylNR⁴(CO)R⁵, O(CO)NR⁴R⁵, NR⁴(CO)OR⁵, NR⁴(CO)NR⁴R⁵, O(CO)OR⁴, O(CO)R⁴, OC₁₋₆alkylCOR⁴, NR⁴(CO)(CO)R⁴, NR⁴(CO)(CO)NR⁴R⁵, SR⁴, C₀₋₆alkyl(SO₂)NR⁴R⁵, OC₁₋₆alkylNR⁴(SO₂)R⁵, OC₀₋₆alkyl(SO₂)NR⁴R⁵, C₀₋₆alkyl(SO)NR⁴R⁵, OC₁₋₆alkyl(SO)NR⁴R⁵, SO₃R⁴, C₁₋₆alkylNR⁴(SO₂)NR⁴R⁵, C₀₋₆alkylNR⁴(SO)R⁵, OC₀₋₆alkylNR⁴(SO)R⁵, OC₀₋₆alkylSO₂R⁴, C₀₋₆alkylSO₂R⁴, C₀₋₆alkylSOR⁴, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted by one or more A;
m is 0, 1, 2, 3 or 4;
n is 0, 1, 2, 3, 4 or 5;
R³ is hydrogen, C₁₋₆alkyl, C₂₋₆alkenyl, C₂₋₆alkynyl, C₀₋₆alkylC₃₋₆cycloalkyl, C₁₋₆alkylNR⁶R⁷ or C₁₋₆alkylCONR⁶R⁷;
R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁶R⁷;
R⁴ and R⁵ may together form a 5 or 6 membered heterocyclic ring containing one or more heteroatoms selected from N, O or S, wherein said heterocyclic ring may be optionally substituted by A;
R⁶ and R⁷ are independently selected from hydrogen, C₁₋₆alkyl, C₂₋₆alkenyl, C₂₋₆alkynyl and C₀₋₆alkylC₃₋₆cycloalkyl;
R⁶ and R⁷ may together form a 5 or 6 membered heterocyclic ring containing one or more heteroatoms selected from N, O or S, wherein said heterocyclic ring may be optionally substituted by A;
R⁸ and R⁹ are independently selected from hydrogen, C₁₋₆alkyl, C₂₋₆alkenyl, C₂₋₆alkynyl, C₀₋₆alkylaryl, C₀₋₆alkylheteroaryl and C₀₋₆alkylC₃₋₆cycloalkyl;
R⁸ and R⁹ may together form a 5 or 6 membered heterocyclic ring containing one or more heteroatoms selected from N, O or S, wherein said heterocyclic ring may be optionally substituted by A;
R¹⁴ is hydrogen, methyl, chloro, or bromo;
wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl defined under R³ to R⁹ may be substituted by one or more A;
A is halo, nitro, CHO, CN, OR⁴, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R, NR⁴R⁵, CO₂R⁸, CONR⁴R⁵, NR⁴(CO)R⁴, O(CO)R⁴, COR⁴, SR⁴, (SO₂)NR⁴R⁵, (SO)NR⁴R⁵, SO₃R⁴, SO₂R⁴ or SOR⁴, as a free base or a pharmaceutically acceptable salt thereof, with the proviso that
Y is not methylene or ethylene when both P and Q are phenyl and
Y is not methylene when P is methoxypyrazine and Q is phenyl.

2. A compound of formula I 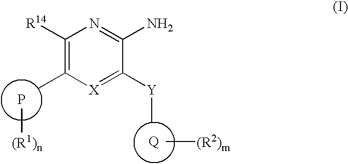
wherein:
Y is CONR³, NR³CO, SO₂NR³, NR³SO₂, CH₂NR³, NR³CH₂, NR³CONR³, CH₂CO, COCH₂, CH═CH, OCH₂ or CH₂O;
X is CH or N;
P is phenyl or a 5 or 6 membered heteroaromatic ring containing one or more heteroatoms selected from N, O or S and said phenyl ring or 5 or 6 membered heteroaromatic ring may optionally be fused with a 5 or 6 membered saturated, partially saturated or unsaturated ring containing atoms selected from C, N, O or S; .
Q is phenyl or a 5 or 6 membered heteroaromatic ring containing one or more heteroatoms selected from N, O or S wherein at least one atom is nitrogen;
R¹ is halo, nitro, C₀₋₆alkylCN, C₀₋₆alkylOR⁸, fluoromethyl, difluoromethyl, trifluoromethyl, C₀₋₆alkylNR⁸R⁹, C₀₋₆alkylCONR⁸R⁹, C₀₋₆alkylNR⁸(CO)R⁹, NR⁸(CO)OR⁹, C₀₋₆alkylO(CO)R⁸, C₀₋₆alkylSO₂R⁸, C₀₋₆alkylSOR⁸, C₀₋₆alkylCOR⁸, C₀₋₆alkylO(CO)OR⁸, OC₀₋₆alkylSO₂R⁸, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted on any carbon atom by one or more A; and if said heteroaryl contains a —NH— moiety that nitrogen may be optionally substituted by A;
R² is halo, nitro, CHO, C₀₋₆alkylCN, OC₁₋₆alkylCN, C₀₋₆alkylOR⁴, OC₁₋₆alkylOR⁴, fluoromethyl, difluoromethyl, trifluoromethyl, fluoromethoxy, difluoromethoxy, trifluoromethoxy, C₀₋₆alkylNR⁴R⁵, OC₁₋₆alkylNR⁴R⁵, OC₁₋₆alkylOC₁₋₆alkylNR⁴R⁵, NR⁴OR⁵ C₀₋₆alkylCO₂R⁴, OC₁₋₆alkylCO₂R⁴, C₀₋₆alkylCONR⁴R⁵, OC₁₋₆alkylCONR⁴R⁵, OC₁₋₆alkylNR⁴(CO)R⁵, C₀₋₆alkylNR⁴(CO)R⁵, O(CO)NR⁴R⁵, NR⁴(CO)OR⁵, NR⁴(CO)NR⁴R⁵, O(CO)OR⁴, O(CO)R⁴, OC₁₋₆alkylCOR⁴, NR⁴(CO)(CO)R⁴, NR⁴(CO)(CO)NR⁴R⁵, SR⁴, C₀₋₆alkyl(SO₂)NR⁴R⁵, OC₁₋₆alkylNR⁴(SO₂)R⁵, OC₀₋₆alkyl(SO₂)NR⁴R⁵, C₀₋₆alkyl(SO)NR⁴R⁵, OC₁₋₆alkyl(SO)NR⁴R⁵, SO₃R⁴, C₁₋₆alkylNR⁴(SO₂)NR⁴R⁵, C₀₋₆alkylNR⁴(SO)R⁵, OC₀₋₆alkylNR⁴(SO)R⁵, OC₀₋₆alkylSO₂R⁴, C₀₋₆alkylSO₂R⁴, C₀₋₆alkylSOR⁴, C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl, wherein any C₁₋₆alkyl, C₂₋₆alkenyl, C₂₋₆alkynyl, C₀₋₆alkylC₃₋₆cycloalkyl, C₀₋₆alkylaryl or C₀₋₆alkylheteroaryl may be optionally substituted on any carbon atom by one or more A, and if said heteroaryl contains a —NH— moiety that nitrogen may be optionally substituted by A;
m is 0, 1, 2, 3 or 4;
n is 0, 1, 2, 3, 4 or 5;
R³ is hydrogen, C₁₋₆alkyl, C₂₋₆alkenyl, C₂₋₆alkynyl, C₀₋₆alkylC₃₋₆cycloalkyl, C₁₋₆alkylNR⁶R⁷ or C₁₋₆alkylCONR⁶R⁷;
R⁴ and R⁵ are independently selected from hydrogen, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl and C₁₋₆alkylNR⁶R⁷;
R⁴ and R⁵ may together form a 5 or 6 membered heterocyclic ring containing one or more heteroatoms selected from N, O or S, wherein if said heterocyclic ring contains an —NH— moiety that ring nitrogen may be optionally substituted by A;
R⁶ and R⁷are independently selected from hydrogen, C₁₋₆alkyl, C₂₋₆alkenyl, C₂₋₆alkynyl and C₀₋₆alkylC₃₋₆cycloalkyl;
R⁶ and R⁷ may together form a 5 or 6 membered heterocyclic ring containing one or more heteroatoms selected from N, O or S, and if said heterocyclic ring contains a —NH— moiety that ring nitrogen may be optionally substituted by A;
R⁸ and R⁹ are independently selected from hydrogen, C₁₋₆alkyl, C₂₋₆alkenyl, C₂₋₆alkynyl, C₀₋₆alkylaryl, C₀₋₆alkylheteroaryl and C₀₋₆alkylC₃₋₆cycloalkyl;
R⁸ and R⁹ may together form a 5 or 6 membered heterocyclic ring containing one or more heteroatoms selected from N, O or S, and if said heterocyclic ring contains a —NH— moiety that ring nitrogen may be optionally substituted by A;
R¹⁴ is hydrogen;
wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl defined under R³ to R⁹ may be substituted by one or more A;
A is halo, nitro, CHO, CN, OR⁴, C₁₋₆alkyl, C₂₋₆alkenyl, C₂₋₆alkynyl, C₀₋₆alkylC₃₋₆cycloalkyl, fluoromethyl, difluoromethyl, trifluoromethyl, fluoromethoxy, difluoromethoxy, trifluoromethoxy, C₀₋₆alkylNR⁴R⁵, OC₁₋₆-alkylNR⁴R⁵, NR⁴R⁵, CO₂R⁴, CONR⁴R⁵, NR⁴(CO)R⁴, O(CO)R⁴, COR⁴, SR⁴, (SO₂)NR⁴R⁵, (SO)NR⁴R⁵, SO₃R⁴, SO₂R⁴ or SOR⁴, as a free base or a pharmaceutically acceptable salt thereof.

3. A compound according to any one of claims 1 and 2 wherein:
Y is CONR³;
X is N;
P is phenyl or a 5 membered heteroaromatic ring containing one heteroatom selected from O or S and said phenyl ring may optionally be fused with a 5 membered saturated ring containing atoms selected from C or O;
Q is a pyridine;
R¹ is halo, nitro, C₀₋₆alkylCN, C₀₋₆alkylOR⁸; trifluoromethyl, C₀₋₆alkylCONR⁸R⁹, C₁₋₆alkyl, C₁₋₆alkylCO₂R⁸, C₀₋₆alkylOR⁴ or C₀₋₆alkylNR⁴R⁵;
m is 0 or 1;
n is 0, 1 or 2;
R³ is hydrogen;
R⁴ and R⁵ are hydrogen;
R⁴ and R⁵ may together form a 5 membered heterocyclic ring containing one heteroatom selected from N;
R⁸ and R⁹ are hydrogen;
R¹⁴ is hydrogen or methyl.

4. A compound according to any one of claims 1 to 3, wherein Y is CONR³.

5. A compound according to any one of claims 1 to 4, wherein P is phenyl.

6. A compound according to any one of claims 1 to 4, wherein P is a 5 or 6 membered heteroaromatic ring containing heteroatoms selected from N, O or S.

7. A compound according to claim 6, wherein P is furan or thiophene.

8. A compound according to any one of claims 1 to 7, wherein Q is pyridine.

9. A compound which is
3-Amino-6-phenyl-N-pyridin-3-ylpyrazine-2-carboxamide,
3-Amino-6-(2-methylphenyl)-N-pyridin-3-ylpyrazine-2-carboxamide,
3-Amino-6-(4-cyanophenyl)-N-pyridin-3-ylpyrazine-2-carboxamide,
3-Amino-6-(3,4-methylenedioxyphenyl)-N-pyridin-3-ylpyrazine-2-carboxamide,
3-Amino-6-(2-thienyl)-N-pyridin-3-ylpyrazine-2-carboxamide,
3-Amino-6-(3-nitrophenyl)-N-pyridin-3-ylpyrazine-2-carboxamide,
3-Amino-6-(3,5-bistriflouromethylphenyl)-N-pyridin-3-ylpyrazine-2-carboxamide,
3-Amino-6-(3-thienyl)-N-pyridin-3-ylpyrazine-2-carboxamide,
3-Amino-6-(4-fluorophenyl)-N-pyridin-3-ylpyrazine-2-carboxamide,
3-Amino-6-(4-chlorophenyl)-N-pyridin-3-ylpyrazine-2-carboxamide,
3-Amino-6-(2,3-dichlorophenyl)-N-pyridin-3-ylpyrazine-2-carboxamide,
3-Amino-6-(2,4-dichlorophenyl)-N-pyridin-3-ylpyrazine-2-carboxamide,
3-Amino-6-(2,4-difluorophenyl)-N-pyridin-3-ylpyrazine-2-carboxamide,
3-Amino-6-(3,4-difluorophenyl)-N-pyridin-3-ylpyrazine-2-carboxamide,
3-Amino-6-(3-chloro-4-fluorophenyl)-N-pyridin-3-ylpyrazine-2-carboxamide,
3-Amino-6-[4-fluoro-3-methylphenyl]-N-pyridin-3-ylpyrazine-2-carboxamide,
3-Amino-6-(3,4-dimethylphenyl)-N-pyridin-3-ylpyrazine-2-carboxamide,
3-Amino-6-(3-fluorophenyl)-N-pyridin-3-ylpyrazine-2-carboxamide or
3-Amino-6-(2-fluorophenyl)-N-pyridin-3-ylpyrazine-2-carboxamide as a free base or a pharmaceutically acceptable salt thereof.

10. A compound which is
3-Amino-6-(2,4-dichlorophenyl)-N-(4-(2-pyrrolidin-1-ylethyl)pyridin-3-yl]pyrazine-2-carboxamide,
3-Amino-6-(3-chloro-4-fluorophenyl)-N-[4-(2-pyrrolidin-1-ylethyl)pyridin-3-yl]pyrazine-2-carboxamide,
3-Amino-6-(2-furyl)-N-pyridin-3-ylpyrazine-2-carboxamide,
3-Amino-6-(4-hydroxyphenyl)-N-pyridin-3-ylpyrazine-2-carboxamide, or
3-Amino-6-[4-(aminocarbonyl)phenyl]-N-pyridin-3-ylpyrazine-2-carboxamide as a free base or a pharmaceutically acceptable salt thereof, or
3-Amino-6-(2,4-dichlorophenyl)-N-(4-(2-pyrrolidin-1-ylethyl)pyridin-3-yl]pyrazine-2-carboxamide hydrochloride,
3-Amino-6-(3-chloro-4-fluorophenyl)-N-[4-(2-pyrrolidin-1-ylethyl)pyridin-3-yl]pyrazine-2-carboxamide hydrochloride or
3-Amino-6-(4-hydroxyphenyl)-N-pyridin-3-ylpyrazine-2-carboxamide hydrochloride.

11. A compound which is
3-Amino-6-(4-chlorophenyl)-5-methyl-N-pyridin-3-ylpyrazine-2-carboxamide or 4-{5-Amino-6-[(pyridin-3-ylamino)carbonyl]pyrazin-2-yl} benzoic acid as a free base or a pharmaceutically acceptable salt thereof.

12. A pharmaceutical formulation comprising as active ingredient a therapeutically effective amount of the compound of any one of claims 1 to 11 in association with pharmaceutically acceptable diluents, excipients or inert carriers.

13. The pharmaceutical formulation according to claim 12 for use in the prevention and/or treatment of conditions associated with glycogen synthase kinase-3.

14. The pharmaceutical formulation according to claim 12 for use in the prevention and/or treatment of Parkinson's Disease, Frontotemporal dementia Parkinson's Type, Parkinson dementia complex of Gaum, HIV dementia diseases with associated neurofibrillar tangle pathologies, amyotrophic lateral sclerosis, corticobasal degeneration, dementia pugilistica, Down syndrome, Huntington's Disease, postencephelatic parkinsonism, progressive supranuclear palsy, Pick's Disease Niemann-Pick's Disease, stroke, head trauma and other chronic neurodegenerative diseases, Bipolar Disorder, affective disorders, depression, schizophrenia, cognitive disorders, Type I and Type II diabetes, diabetic neuropathy, hair loss or contraceptive medication.

15. The pharmaceutical formulation according to claim 12, for use in the prevention and/or treatment of dementia or Alzheimer's Disease.

16. The pharmaceutical formulation according to claim 12, for use in the prevention and/or treatment of diabetes.

17. A compound as defined in any one of claims 1 to 11 for use in therapy.

18. The compound as defined in claim 17 for use in prevention and/or treatment of conditions associated with glycogen synthase kinase-3.

19. The compound as defined in claim 17 for use in prevention and/or treatment of Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, postencephelatic parkinsonism, progressive supranuclear palsy, Pick's Disease, Niemann-Pick's Disease, stroke, head trauma and other chronic neurodegenative diseases, Bipolar Disorder, affective disorders, depression, schizophrenia, cognitive disorders, Type I and Type II diabetes, diabetic neuropathy, hair loss and contraceptive medication.

20. The compound as defined in claim 17, for use in prevention and/or treatment of dementia or Alzheimer's Disease.

21. A compound as defined in claim 17, for use in prevention and/or treatment of diabetes.

22. The use of a compound defined in any one of claims 1 to 11 in the manufacture of a medicament for the use in the prevention and/or treatment of conditions associated with glycogen synthase kinase-3.

23. The use of a compound as defined in any of claims 1 to 11 in the manufacture of a medicament for the prevention and/or treatment of Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, postencephelatic parkinsonism, progressive supranuclear palsy, Pick's Disease, Niemann-Pick's Disease, stroke, head trauma and other chronic neurodegenative diseases, Bipolar Disorder, affective disorders, depression, schizophrenia, cognitive disorders, Type I and Type II diabetes, diabetic neuropathy, hair loss and contraceptive medication.

24. The use of a compound as defined in any of claims 1 to 11, in the manufacture of a medicament for the prevention and/or treatment of dementia or Alzheimer's Disease.

25. The use of a compound as defined in any of claims 1 to 11, in the manufacture of a medicament for the prevention and/or treatment of diabetes.

26. A method of prevention and/or treatment of conditions associated with glycogen synthase kinase-3, comprising admirnistrering to a mammal, including man in need of such prevention and/or treatment, a therapeutically effective amount of a compound of formula I as defined in any one of claims 1 to 11.

27. A method of prevention and/or treatment of Parkinson's Disease, Frontoterhporal dementia Parkinson's Type, Parkinson dementia complex of Gaum; HIV dementia, diseases with associated neurofibrillar tangle pathologies, amyotrophic lateral sclerosis, corticobasal degeneration, dementia pugilistica, Down syndrome, Huntington's Disease, postencephelatic parkinsonism, progressive supranuclear palsy, Niemann-Pick's Disease, Pick's Disease, stroke, head trauma and other chronic neurodegenative diseases, Bipolar Disorder, affective disorders, depression, schizophrenia, cognitive disorders, Type I and Type II diabetes, diabetic neuropathy, hair loss and contraceptive medication comprising administrering to a mammal, including man in need of such prevention and/or treatment, a therapeutically effective amount of a compound of formula I as defined in any one of claims 1 to 11.

28. A method of prevention and/or treatment of dementia or Alzheimer's Disease comprising administrering to a mammal, including man in need of such prevention and/or treatment, a therapeutically effective amount of a compound of formula I as defined in any one of claims 1 to 11.

29. A method of prevention and/or treatment of diabetes comprising administrering to a mammal, including man in need of such prevention and/or treatment, a therapeutically effective amount of a compound of formula I as defined in any one of claims 1 to 11.

30. Processes for the preparation of a compound of the formula I, wherein Y, X, P, Q, R¹, R², R³, R⁴, R⁵, R⁶, R⁷, R⁸, R⁹, R¹⁴, A, m and n are, unless specified defined as in formula I according to any one of claims 1 to 3, comprising of:
A) a de-halogen coupling of a compound of formula IV with an aryl species to give a compound of formula I: 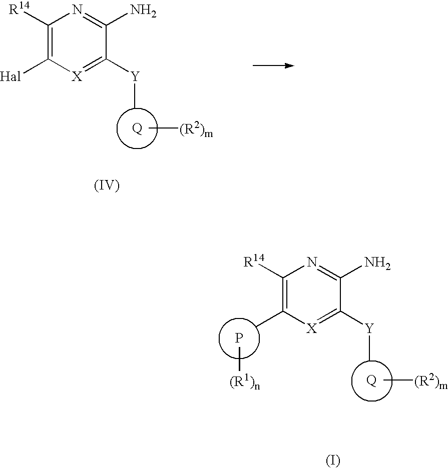
B) amidation of a compound of formula VI with an appropriate amine: 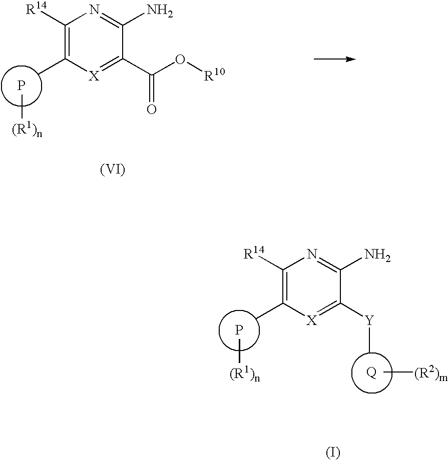
C) de-halogen coupling of a compound of formula VIII with an aryl species to give a compound of formula I: 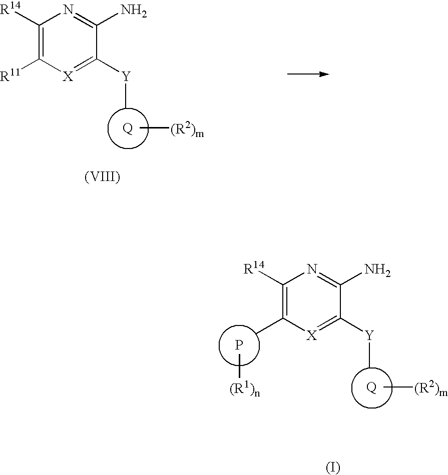
D) amidation of a compound of formula XII with an appropriate amine: 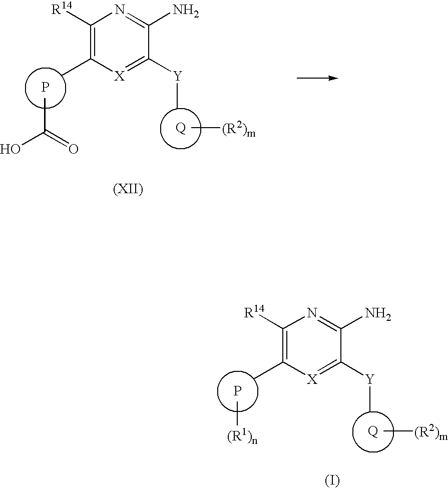
wherein an aryl species in route A and C is selected from aryl halogen, aryl boronic acid and aryl stannane,
and an appropriate amine in route B and D is selected from a compound of formula XI, HNR⁸R⁹ or 3-aminopyridine.

31. A compound of formula VI 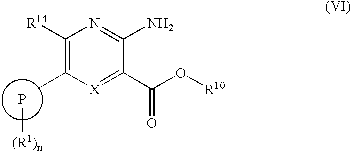
wherein X, P, R¹, R⁸, R⁹, R¹⁴, A and n are defined as in formula I according to any one of claims 1 to 3 and R¹⁰ is hydrogen or C₁₋₆alkyl, with the proviso that
i) when P is phenyl then R¹⁰ is C₃alkyl;
ii) when P is 4-chlorophenyl then R¹⁰ is C₂₋₆alkyl;
iii) when P is 4-methoxyphenyl then R¹⁰ is hydrogen or C₂₋₆alkyl;
iv) when P is pyridine then R¹⁰ cannot be methyl, ethyl or n-butyl;
v) when P is furan or benzothienyl then R¹⁰ cannot be methyl.

32. A compound according to claim 31 wherein P is phenyl and R¹⁰ is C₃₋₆alkyl.

33. A compound according to claim 31 wherein P is furan and R¹⁰ is C₂₋₆alkyl.

34. A compound according to claim 31 wherein P is thiophene.

35. A compound of formula IV 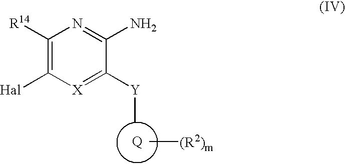
wherein X, R², R⁴, R⁵, R⁶, R⁷, A and m are defined as in formula I according to any one of claims 1 to 3 and R¹⁴ is hydrogen or methyl.

36. A compound which is
3-Amino-6-bromo-N-pyridin-3-ylpyrazine-2-carboxamide,
3-Amino-6-bromo-5-methyl-N-pyridin-3-ylpyrazine-2-carboxamide,
tert-Butyl 4-(2-hydroxyethyl)pyridin-3-ylcarbamate,
tert-Butyl 4-(2-pyrrolidin-1-ylethyl)pyridin-3-ylcarbamate,
4-(2-Pyrrolidin-1-ylethyl)pyridin-3-amine or
3-Amino-6-bromo-N-[4-(2-pyrrolidin-1-ylethyl)pyridine-3-yl]pyrazine-2-carboxamide.

37. A compound according to any of claims 31 to 36, which can be used as an intermediate in the preparation of a compound of formula I according to any one of claims 1 to 11.
